(19) United States
(12) Patent Application Publication
(10) Pub. No.: US 2026/0317814 A1
(43) Pub. Date: Oct. 15, 2026

(54) Compositions and Methods for Cell Type-Specific CRISPR-Based Genetic Screening

(71) Applicant: The Regents of the University of California, Oakland, CA (US)

(72) Inventors: Martin Kampmann, San Francisco, CA (US); Andrew Pan, San Francisco, CA (US); Biswarathan Ramani, San Francisco, CA (US); Indigo Rose, San Francisco, CA (US)

(21) Appl. No.: 19/473,955

(22) PCT Filed: Apr. 10, 2024
(86) PCT No.: PCT/US2024/023843

§ 371 Date: Oct. 9, 2025

Related U.S. Application Data
(60) Provisional application No. 63/495,956, filed on Apr. 13, 2023.

(51) Int. Cl. C12Q 1/6844; C12N 9/22; C12N 15/113; C12Q 1/48; C12Q 1/6806; C12Q 1/6813; C12Q 1/6869; C12Q 1/6876; C12Q 1/6897; C40B 30/00; C40B 40/06

(52) U.S. Cl. CPC C12Q 1/6844; C12N 9/226; C12N 15/113; C12Q 1/48; C12Q 1/6806; C12Q 1/6813; C12Q 1/6869; C12Q 1/6876; C12Q 1/6897; C12Y 207/07; C40B 30/00; C40B 40/06; C12N 2310/20

(57) ABSTRACT

Provided are compositions and methods for verifying the introduction of a CRISPR/Cas guide RNA into a target cell of interest. Also provided are compositions and methods for CRISPR-based functional screening in vivo in non-human subjects. Such methods can be used to identify target sequences (e.g., genes) of interest that play roles in cell survival, cell death, cell proliferation, and the like, or any other phenotypes measurable by CRISPR/Cas guide RNA-based screening. A subject method includes introducing a nucleic acid that encodes a CRISPR/Cas guide RNA and that includes an inversion cassette into a target cell expressing a recombinase, where the recombinase catalyzes inversion of the cassette. An amplification reaction is performed using a primer pair in which one primer is specific to the inverted sequence. Libraries of such guide RNA-expressing AAVs can be introduced, and guide RNA depletion can be determined using such an amplification reaction.

### CROSS-REFERENCE

This application claims the benefit of U.S. Provisional Patent Application No. 63/495,956 filed Apr. 13, 2023, which application is incorporated herein by reference in its entirety.

### INCORPORATION BY REFERENCE OF SEQUENCE LISTING PROVIDED AS AN XML FILE

A Sequence Listing is provided herewith as a Sequence Listing XML, “UCSF-716WO_SeqList.xml” created on Mar. 20, 2024 and having a size of 26,463 bytes. The contents of the Sequence Listing XML are incorporated by reference herein in their entirety.

### I. INTRODUCTION

Pooled CRISPR-based genetic screens have become a powerful tool for unbiased biological discovery and hypothesis generation. CRISPR screens in general are performed in immortalized cell lines that take advantage of lentiviral single guide RNA (sgRNA) libraries that integrate into the genome, followed by selection and expansion of the transduced cells. After further selective pressure for a phenotype of interest, the sgRNAs are recovered from the bulk genomic DNA for downstream sequencing and analysis.

While CRISPR screening has been immensely successful in cell culture systems, they have been challenging to perform in tissues of living organisms. Mice are among the most widely used mammalian model system to understand cell-type-specific process in a physiological in vivo context. There have been rare applications of CRISPR screening performed in mice¹⁻³, including in the brain, using direct lentiviral delivery of a pooled sgRNA library. However, lentiviral-based delivery of sgRNAs into tissues suffer from several significant limitations. One, there are few types of lentiviral capsids and offers little control on infectivity of different tissues and cell-types. Two, lentivirus injected into a tissue has limited spread beyond the region of injection. Three, although lentiviral genomic integration allows greater representation of an sgRNA through clonal proliferation, this does not offer an advantage for non-dividing cells, such as neurons. Along these lines, recovery of small number of sgRNAs from bulk genomic DNA from tissue is time- and resource-consuming, and would require an extraordinary amount of PCR reagents to amplify the sgRNAs.

Adeno-associated virus (AAV) has rapidly emerged as a robust way to deliver genes to the brain. Compared to lentivirus, they show far better spread through the mouse brain and targets of large proportion of neurons and other brain cell types. There are a growing number AAV capsids being routinely optimized and improved for targeting of different cell types, along with additional options for intravenous delivery rather than direct injection of a target organ⁴. AAVs do not integrate into the host genome but are maintained as a circular episome within the cell. Although this would be a disadvantage for highly proliferative immortalized cell lines, it would not be a major limitation for nondividing differentiated tissues of an adult organism. AAV episomes isolated from mouse brain homogenates at the endpoint of the screen vastly increases the scalability of screens compared to lentivirus-based approaches⁵, in which sgRNA sequences are amplified from total genomic DNA. However, a significant limitation to both lentiviral- and AAV-based strategies is that when sgRNA sequences are recovered from tissues, it would not discriminate between different cell-types or from DNA within the virions that may not have entered cells.

Provided are methods and compositions that combine AAV and enzyme-dependent sgRNA recovery screening, facilitating tissue-based screening in non-human subjects in vivo, e.g., in mice. The illustrative working examples described below demonstrate identification of neuron essential genes within a mouse brain.

### II. SUMMARY

The inventors have designed and exemplified a robust method of verifying the introduction of a CRISPR/Cas guide RNA into a target cell of interest. The inventors also realized and exemplified that such a method can be used (by employing a library of CRISPR/Cas guide RNAs), to perform robust in vivo functional screens to identify endogenous sequences that play roles in controlling various downstream phenotypes such as cell number (e.g., sequences that play a role in cell survival, cell death, cell proliferation, and the like), protein levels, transcriptomic outcomes, or any other phenotype measurable by CRISPR/Cas guide RNA-based screening. Toxins, drugs, or disease models can be used to apply additional selective pressure.

As such, provided are compositions and methods for verifying the introduction of a CRISPR/Cas guide RNA into a target cell of interest. Also provided are compositions and methods for CRISPR-based functional screening in vivo in non-human subjects. Such methods can be used to identify target sequences (e.g., genes) of interest that play roles in cell survival, cell death, cell proliferation, and the like, or any other phenotype measurable by CRISPR/Cas guide RNA-based screening. As noted above, toxins, drugs, or disease models can be used to apply additional selective pressure.

In general, a subject method of verifying the introduction of a CRISPR/Cas guide RNA into a target cell of interest includes introducing a nucleic acid encoding a CRISPR/Cas guide RNA into the target cell of interest, extracting DNA (e.g., episomal DNA) from the target cell, and performing an amplification reaction with a primer pair to determine whether the nucleic acid encoding a CRISPR/Cas guide RNA had actually entered the cell.

To do this, the target cell of interest expresses a recombinase protein (e.g., a tyrosine-type site-specific recombinase (YSSR) such as Cre recombinase, a serine recombinase such as phiC31, and the like) that inverts a probe sequence (e.g., a spacer sequence) on the nucleic acid encoding the CRISPR/Cas guide RNA. The probe sequence is therefore only inverted in cells expressing the recombinase protein (i.e., target cells of interest). The probe sequence is inverted by the recombinase because it is positioned between target sites for the recombinase protein (e.g., Lox sites for Cre recombinase, FRT sites for Flp recombinase, attB and attP sites for phiC31, and the like). In short, the inversion establishes a record of a cell having received the guide RNA and having expressed/contained the active recombinase (e.g., Cre).

In some embodiments, the probe sequence is a “spacer sequence”, an additional sequence that is not part of the guide RNA expression cassette (i.e., not part of the CRISPR/Cas guide RNA or the promoter to which it is operably linked). In some cases, the probe sequence can include the promoter that controls the expression of the guide RNA (e.g., a pol III promoter such as a U6 promoter, an H1 promoter, and the like). In some cases, the probe sequence can include the CRISPR/Cas guide RNA in an improper orientation relative to its promoter prior to inversion such that the guide RNA would only be expressed by the promoter after inversion. In some cases, the probe sequence can include both the CRISPR/Cas guide RNA and the promoter to which it is operably linked.

In some cases, the recombinase catalyzes a deletion/excision instead of an inversion, generating a new sequence (a “neo-sequence”) that functions as a probe sequence for amplification.

In some cases the recombinase is a Cre or Flp recombinase and the target sites are Lox or FRT sites, respectively. In some such cases, the Lox or FRT sites are mutant sites that provided for unidirectional recombination. In some cases, the spacer sequence has a length of from 100-400 base pairs. In some cases, the recombinase is a serine recombinase, e.g., a unidirectional serine recombinase such as phiC31 and the target sites are attB/attP sites.

In some cases, the target cell is a non-dividing cell. In some cases, the target cell is a neuron. In some cases, the target cell is a brain cell. In some cases, the target cell is a non-neuronal brain cell, such as a glial cell.

To detect the inversion, one of the two primers used in the amplification reaction hybridizes to a sequence within the probe sequence (e.g., spacer sequence) such that the two primers do not form a primer pair prior to the inversion (i.e., they hybridize to the same strand of the double stranded target DNA prior to the inversion), but do form a primer pair after the inversion (i.e., they hybridize to opposite strands of the double stranded target DNA after inversion of the probe sequence). A positive result from the amplification reaction—an amplified product of the expected size (or sequence) —indicates that guide RNA entered the cell (because the nucleic acid was exposed to a recombinase present within the cell).

Moreover, if the sequence encoding the guide sequence of the CRISPR/Cas guide RNA is positioned within the introduced nucleic acid such that it is between the two primers of the primer pair (after inversion of the probe sequence), then the amplification product of the amplification reaction can be sequenced and thereby used to identify the guide sequence. As such, this verification method can be used for functional screens in vivo in non-human subjects.

For CRISPR-based in vivo screening methods in non-human subjects, a library of nucleic acids encoding CRISPR/Cas guide RNAs (where the guide RNAs of the members of the library have different guide sequences from one another) are introduced. Any convenient delivery method can be used (e.g., lentivirus, nanoparticles, lipid nanoparticles, AAV, and the like), e.g., via AAV infection to a population of target cells (e.g., brain cells). When AAV infection is used, to introduce the guide RNAs, any convenient AAV capsid can be used and it will vary depending on the targeted cell/tissue/organ type. For example, in some cases, transduction of neurons through intravenous delivery is desired and an AAV-PHP.eB capsid may be used. In some cases, other viral types such as lentivirus, rabies virus, polio virus, etc. are used for delivery. In some cases, non-viral methods of delivery are used, e.g., nanoparticles such as lipid nanoparticles.

In some cases, after introducing a guide RNA library into a population of target cells (e.g., contacting the population of target cells with a guide RNA library such as an AAV guide RNA library), a period of time is allowed to pass (the “wait time”) prior to obtaining the cells for DNA extraction (e.g., episomal DNA). This allows the cells time to develop/exhibit a phenotype due to expression of the guide RNAs. In some cases, a biological sample (one that includes the contacted population of target cells) is collected from the subject in a range of from 1 day to 6 months (e.g., 1-8 weeks, 2-7 weeks, 3-6 weeks, about 4 weeks, about 5 weeks, about 6 weeks, about 7 weeks, or about 8 weeks) after introducing the library. The biological sample can be from any tissue or organ (e.g., brain, heart, kidney, liver, lung, pancreas, retina, or skeletal muscle), and in some cases the sample is the whole organ.

In some cases, such as CRISPR/Cas guide RNA-based screens designed to measure any phenotype beyond cell survival, cell death, cell proliferation, and the like, cells are isolated from the collected tissue or organ and sorted. Cells may be physically sorted into multiple different populations which are treated as separate samples during DNA extraction and amplification. Comparing the guide RNA frequencies in these different samples allows the detection of guide RNAs which alter any detectable cellular phenotype. Detecting a cellular phenotype may include using an antibody, dye, or engineered fluorescence-based reporter to detect any protein, lipid, carbohydrate, or other molecular target and subsequently sorting using fluorescence-activated cell sorting (FACS) or magnetic-activated cell sorting (MACS) or similar technologies to read out the screening phenotype. It may also include using single cell RNA sequencing (scRNA-seq) or single nucleus RNA sequencing (snRNA-seq) or similar technologies to read out the screening phenotype in individual cells or nuclei, along with corresponding transcriptomic information or other information at the single cell or nucleus level.

Once the DNA is extracted and the amplification reaction performed, the amplification product (a sample of amplified guide sequences) should include guide sequences for all guide RNAs still present in the target cell population. For example, for guide RNAs that lead to cell death over the course of the ‘wait time’, those cells will not be present in the cell population by the time cells are obtained and DNA extracted—and therefore those guide sequences will not be present in the amplification product. Thus, in some embodiments, a subject method includes a step of detecting and measuring quantities of guide sequences present in the amplification product. The result of said detecting and measuring (a ‘test set of measured values’) can then be compared to a control (a ‘control set of measured values’) to determine whether any of the guide sequences are enriched or depleted in the test set relative to the control set. The control set should provide quantities (e.g., quantities of the different guide sequences relative to one another) that were present in library (e.g., library of AAVs) prior to said contacting. In other words, the amplification product can be assayed (e.g., via high throughput/nextgen sequencing) to determine the relative abundance of the different guide sequences present after contacting the cell population compared to the relative abundance that had been present prior the contacting. Alternatively, the control set can be amplified from cells which did make contact with the guide RNA library, but which lack the CRISPR/Cas effector protein (or lack the ability to activate it). Such a control set may be an organism which lacks the DNA sequences in its genome for creating the CRISPR/Cas effector protein, or an organism whose control of creating the CRISPR/Cas effector protein in activated by a regulatory mechanism, such as mice with a lox-stop-lox-driven CRISPR/Cas effector protein which did not receive the virus for cre-recombinase.

The nature of the CRISPR/Cas effector protein will affect the interpretation of the results of such screening. For example, in some cases the CRISPR/Cas effector protein (e.g., Cas9), is fused to a transcription repressor (i.e., CRISPRi) (e.g., KRAB (Krüppel-associated box)). In such cases, a guide sequence that targets a protein essential for cell survival should lead to decreased expression of the cell survival protein and perhaps cell death, which would result in depletion/reduction of that guide sequence from the amplification product. Likewise, in some cases, the CRISPR/Cas effector protein (e.g., Cas9) is not fused to a heterologous protein but is instead a nickase or fully functional nuclease that creates double strand breaks. In such cases, a guide sequence that leads to knockout (e.g., via non-homologous end joining) of a protein essential for cell survival should lead to decreased expression of the cell survival protein and perhaps cell death, which would result in depletion/reduction of that guide sequence from the amplification product.

On the other hand, in some cases the CRISPR/Cas effector protein (e.g., Cas9) is fused to a transcription activator (i.e., CRISPRa) (e.g., VP64, e.g., using a synergistic activation mediator (SAM) system). In such cases, a guide sequence that targets a protein that is toxic at high levels should lead to increased expression of the toxic protein and perhaps cell death, which would result in depletion/reduction of that guide sequence from the amplification product.

Thus, in some embodiments, the CRISPR/Cas effector protein (e.g., Cas9) is fused to a transcription repressor (i.e., CRISPRi) (e.g., KRAB). In some embodiments, the CRISPR/Cas effector protein (e.g., Cas9) is fused to a transcription activator (i.e., CRISPRa) (e.g., VP64, e.g., using a synergistic activation mediator (SAM) system). In some embodiments, the CRISPR/Cas effector protein (e.g., Cas9) is not fused to a heterologous protein such as with CRISPRi or CRISPRa, but is instead a nickase or fully functional nuclease that creates double strand breaks.

In some cases the subject is an adult, in some cases a juvenile, and in some cases a newborn/neonate. In some cases the subject is a rodent (e.g., rat, mouse). In some embodiments, a library (e.g., AAV library) is introduced into the subject via intravenous administration. In some cases, a library (e.g., AAV library) is introduced into the subject via direct injection to an organ of the subject (e.g., intracerebroventricular (ICV) injection for delivery to the brain).

Because a subject functional screen depends on the presence of two proteins (a CRISPR/Cas effector protein and the recombinase protein for inversion of sgRNAs constructs, which is a prerequisite for their amplification during screen analysis), only cells of interest that express both proteins will be evaluated.

In some cases, the target cells of interest (those expressing a recombinase protein such as Cre) are a subpopulation of the target cells that are infected with a library (e.g., AAV library). For example, in some cases the population of target cells might be brain cells, but the only brain cells of interest are neurons. In such cases, the recombinase protein (e.g., a YSSR such as Cre, or a serin recombinase such as phiC31) can be expressed from a tissue specific or cell type specific promoter, e.g., a neuron specific promoter—thus, the amplification reaction will only detect guide RNA sequences present in those recombinase-expressing cells because those are the only cells in which the probe sequence (e.g., spacer sequence) was inverted and therefore the only cells for which the two primers will form a primer pair. In such cases, the CRISPR/Cas effector protein should be expressed from the same promoter as the recombinase protein, or from a promoter that expresses in the same cells plus additional cells, e.g., a tissue non-specific promoter such as a ubiquitous promoter (e.g., EF1a, CMV, b-actin, and the like).

In some cases, the recombinase protein is already expressed in the target cells of interest before the library is introduced, e.g., a subject such as a recombinase-expressing mouse can be used (e.g., a Cre-expressing mouse). The recombinase can be expressed from any convenient promoter, e.g., tissue-specific promoter (such as a neuron-specific promoter), a temporally controlled promoter, an inducible promoter, a ubiquitous promoter, and the like. Likewise, the recombinase can be introduced or (or its expressed induced) just before or along with the library. Any convenient delivery method can be used (e.g., lentivirus, nanoparticles, lipid nanoparticles, AAV, and the like). In some cases, the recombinase protein is introduced via AAV, e.g., a second AAV (in addition to guide RNA library AAVs) can be used.

Similarly, in some cases, the CRISPR/Cas effector protein is already expressed in the target cells of interest before the library is introduced, e.g., a subject such as a recombinase-expressing mouse can be used (e.g., a Cas9-expressing mouse). The CRISPR/Cas effector protein can be expressed from any convenient promoter, e.g., tissue or cell-type-specific promoter (such as a neuron-specific promoter), a temporally controlled promoter, an inducible promoter, a ubiquitous promoter, and the like. Likewise, the CRISPR/Cas effector protein can be introduced or (or its expressed induced) just before or along with the library. Any convenient delivery method can be used (e.g., lentivirus, nanoparticles, lipid nanoparticles, AAV, and the like). In some cases, the CRISPR/Cas effector protein is introduced via AAV, e.g., a second AAV (in addition to guide RNA library AAVs).

As such, in some embodiments, a subject method includes introducing the recombinase protein (e.g., YSSR such as Cre, or a serin recombinase such as phiC31) and/or the CRISPR/Cas effector protein (e.g., Cas9) to the target cell (using any convenient technique). In some cases, target cells are contacted with an AAV that delivers a nucleic acid encoding the recombinase protein (e.g., YSSR such as Cre, or a serin recombinase such as phiC31) and/or the CRISPR/Cas effector protein (e.g., Cas9).

In some cases, prior to introducing the library, the target cell includes a nucleic acid encoding the recombinase protein and/or the CRISPR/Cas effector protein. In some cases, expression of the recombinase protein is controlled by a tissue specific promoter (e.g., a neuron-specific promoter such as an hSyn1 promoter). In some cases, expression of the CRISPR/Cas effector protein is controlled by a tissue specific promoter (e.g., a neuron-specific promoter such as an hSyn1 promoter). In some cases, expression of the recombinase protein is controlled by a ubiquitous promoter. In some cases, expression of the CRISPR/Cas effector protein is controlled by a ubiquitous promoter.

In some embodiments, the nucleic acid that comprises the probe cassette (the inverting probe, e.g., spacer, plus the flanking recombinase target sites) and encodes the CRISPR/Cas guide RNA also includes a cell marker (e.g., a nucleotide sequence encoding fluorescent protein such as GFP (Green Fluorescent Protein), YFP (Yellow), RFP (Red), or BFP (Blue)). In some cases the cell marker is operably linked to a ubiquitous promoter such as an EF1a promoter. In some cases the fluorescent protein is a nuclear localized BFP such as mTagBFP2. Such cell markers can allow for visualization of transfected/transduced (e.g., AAV-transduced) cells. In some cases the cell marker is a drug selectable marker.

Reagents, compositions, and kits/systems that find use in practicing the subject methods are also provided. For example, provided is a nucleic acid vector (e.g., an AAV nucleic acid vector) (and libraries of such vectors) that: (i) encodes a CRISPR/Cas guide RNA operably linked to a pol Ill promoter functional in a eukaryotic cell; (ii) encodes a cell marker operably linked to a promoter functional in the eukaryotic cell; and (iii) comprises an inversion cassette that comprises a probe sequence (e.g., spacer sequence) flanked by target sites for a recombinase protein.

Also provided is a kit, that includes: (1) a nucleic acid vector (e.g., an AAV nucleic acid vector) that: (a) encodes a CRISPR/Cas guide RNA operably linked to a promoter functional in a eukaryotic cell, and (b) includes an inversion cassette (a probe sequence, e.g., a spacer, flanked by target sites for a recombinase); and (2) a first and a second primer, where the first and second primers are specific for (hybridize to) sequences on the same DNA strand of the nucleic acid vector, where one of the primers hybridizes to the probe sequence such that upon inversion of the probe sequence (by the recombinase protein), the first and second primers form a primer pair. In some such cases, the other primer hybridizes to a sequence such that the guide RNA's guide sequence is positioned between the primer pair.

Also provided are libraries of the above nucleic acid vectors (e.g., AAV nucleic acid vectors), where the encoded CRISPR/Cas guide RNAs of the members of the library have different guide sequences from one another.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 AAV backbone for in vivo CRISPR-based genetic perturbations and Cre-dependent episomal recovery from the brain. Top, schematic of a plasmid backbone (in this case pAP215 plasmid backbone) to deliver sgRNAs for CRISPR perturbations. Cre recombinase expression drives inversion of a Lox-flanked cassette. This inversion can be detected using specific primers. Below, transduced AAV episomes from mouse brain. A mouse brain was transduced with AAV expressing AP215 with or without AAV hSyn-GFP-Cre, which drives Cre recombinase expression only in neurons. PCR using primers as depicted in the plasmid schematic reveals a specific PCR product only upon expression of Cre recombinase, confirming recovery and detection of AAV episomes from transduced neurons in vivo.

FIG. 2. Cell-type specific CRISPRi knockdown of an endogenous gene in vivo. AAV with pAP215 with an sgRNA sequence targeting endogenous CREB1 was delivered by intracerebroventricular (ICV) injection into a mouse harboring conditional CRISPRi machinery. Immunofluorescence staining showed knockdown of CREB1 only upon co-expression of AP215 and hSyn-GFP-Cre (black arrows), relative to sgRNA alone (top panels) or GFP-Cre alone (white arrows).

FIG. 3A-3C. Results of a pilot in vivo CRISPRi neuronal survival screen. (FIG. 3A) Schematic of an in vivo neuronal survival screen. AAV preparations containing hSyn-GFP-Cre and a pooled sgRNA library cloned into AP215 were injected ICV into a n=4 neonatal mice with conditional CRISPR interference machinery. After 6 weeks, episomes were purified from whole brains with PCR to amplify sgRNAs with the Cre-inverted cassettes. (FIG. 3B) Sequencing of sgRNAs demonstrated several gene knockdowns associated with decreased neuronal survival, and the strength of the hits strongly correlated between two mice. (FIG. 3C) Heatmap of the 45 most depleted sgRNAs and 15 most enriched sgRNAs, ordered by average sgRNA depletion across replicate mice (n=4), as measured by product score (effect size*−log 10 (p-value)).

FIG. 4. Validation of screen hit in primary neuronal culture. Primary neurons were cultured from conditional CRISPR interference mice and transduced at DIV4 with AAV pAP215 containing either a non-targeting (NTC) sgRNA or an sgRNA targeting Hspa5, coinfected with a constitutive CAG-mScarlet AAV to visualize cell bodies and their processes. Half of the cultures were also coinfected with neuron-specific hSyn1-cre. 14 days after transduction, sgRNAs targeting Hspa5 and coexpressing hSyn1-cre showed marked neuronal death relative to the non-targeting control or the sgRNAs alone. Scale bar is 250 μm.

FIG. 5A-5C. AAV backbone for cell type-specific, cre-dependent sgRNA recovery. FIG. 5A, Strategy for cell-type selective screening. FIG. 5B, The pAP215 AAV sgRNA backbone contains a Lox71/Lox66 flanked “handle” that undergoes inversion only in cells expressing Cre recombinase, allowing for cell type-specific sgRNA recovery by PCR. FIG. 5C, PCR performed on episomes recovered from mouse brains expressing AAV pAP215 with or without hSyn1-Cre-NLS-GFP (Cre) using the primers diagrammed in (B). PCR amplicons from brains of mice injected by ICV at P0 with pPA215 packaged into AAV PHP.eB, with or without co-injection of hSyn1-Cre packaged into AAV PHP.eB. The first three lanes represent samples from uninjected brain, pAP215 alone, and pAP215+hSyn1-Cre shows amplicons generated with Primer A, which captures presence of AAV episomes, but is not inversion specific. The next three lanes represent amplicons from the same samples generated with Primer B, which binds to the lox-flanked recombination site (handle) and will generate amplicons only after cre-mediated handle inversion.

FIG. 6A-6E. Cre-dependent CRISPRi knockdown of endogenous genes in neurons throughout the mouse brain. FIG. 6A, Expression of AAV-pAP215 (sgRNA, based on BFP marker) and AAV-hSyn1-Cre-NLS-GFP (Cre, based on GFP) in a mouse brain three weeks after neonatal intracerebroventricular (ICV) delivery. Scale bar, 1 mm. FIG. 6B, Immunofluorescent staining for neurons (marker: NeuN) and astrocytes (marker: Sox9) in mouse cortex expressing AAV-hSyn1-Cre-NLS-GFP. Scale bar, 25 μm. Overlap of Cre expression with NeuN and Sox9 within a cortical region demonstrate high specificity for neurons and high coverage of neurons. FIG. 6C, sgRNAs targeting Creb1 (sgCreb1) or a non-targeting control (sgNTC) in AAV-pAP215 were delivered with or without AAV hSyn1-Cre-NLS-GFP via ICV injection into neonatal mice with lox-stop-lox CRISPRi machinery. Brains were stained at 3 weeks with a representative area of frontal cortex shown. Scale bar, 50 μm. FIG. 6D, Higher magnification of the boxed regions in the left panels, with white arrows indicating examples of neurons having received both sgCreb1 and Cre, whereas yellow arrows indicate neurons that received Cre without sgCreb1. Scale bar, 10 μm. FIG. 6E, Quantification of Creb1 levels in sgRNA-positive nuclei within a representative region of cortex (mean±s.e.m, n=3 independent mice).

FIG. 7A-7C. in vivo CRISPRi screen for neuron-essential genes in mouse brains. FIG. 7A, Strategy for in vivo CRISPRi screening to identify neuron-essential genes. FIG. 7B, Knockdown phenotypes for 2,269 genes averaged across n=20 mice at 6 weeks of age after ICV injection of an sgRNA library. Genes with an FDR <0.1 are colored blue, and genes within enriched pathways are further color-coded. FIG. 7C, Phenotypes of neuron-essential genes (rows) from (B) with an FDR <0.1 in individual mice (columns), including with Cre recombinases driven under different promoters, and with and without Cre recombinase expression.

FIG. 8A-8D. Validation of Hspa5 as an essential gene in mouse neurons. FIG. 8A, Cultured mouse embryonic fibroblasts (MEFs) from constitutive dCas9-KRAB mice were transduced with lentivirus (pMK1334) containing an sgRNA targeting Hspa5 (sgHspa5) versus a non-targeting control sgRNA (sgNTC). Hspa5 mRNA levels were assayed by qPCR and normalized to the sgNTC control (mean±s.d., n=2 technical replicates). FIG. 8B, Survival of primary neurons cultured from conditional CRISPRi mice following transduction with AAV-hSyn1-Cre-NLS-GFP alongside AAV-AP215 containing sgHspa5 versus sgNTC (mean±s.d., n=4 wells). FIG. 8C, Representative images of primary neurons at 8 or 14 days post transduction with the AAVs as in panel B. Cells were co-transduced with constitutively expressed cytosolic mScarlet, which reveals fine neuronal processes, displayed in this panel. Scale bar 125 μm. FIG. 8D, Representative brains of conditional CRISPRi mice 16 days after neonatal ICV injection of AAV-pAP215-sgHspa5 with or without hSyn1-Cre-NLS-GFP, and brain measurements (mean±s.d., n=3 independent mice). Scale bar, 5 mm.

### IV. DEFINITIONS

“Operatively linked” or “operably linked” refers to a juxtaposition of genetic elements, wherein the elements are in a relationship permitting them to operate in the expected manner. For instance, a promoter is operatively linked to a sequence of interest (the sequence of interest can also be said to be operatively linked to the promoter) if the promoter helps initiate transcription of the sequence of interest. There may be intervening residues between the promoter and sequence of interest so long as this functional relationship is maintained.

“Heterologous” means derived from a genotypically distinct entity from the rest of the entity to it is being compared too. For example, a polynucleotide introduced by genetic engineering techniques into a plasmid or vector derived from a different species is a heterologous polynucleotide. A promoter removed from its native coding sequence and operatively linked to a coding sequence it is not naturally found linked to a heterologous promoter. For example, an AAV including a heterologous nucleic acid encoding a heterologous gene product is an AAV including a nucleic acid not normally included in a naturally occurring, wild-type AAV, and the encoded heterologous gene product is a gene product not normally encoded by a naturally-occurring, wild-type AAV. An AAV including a nucleic acid encoding a variant AAV capsid polypeptide includes a heterologous nucleic acid sequence. Once transferred/delivered into a host cell, a heterologous polynucleotide, contained within the virion, can be expressed (e.g., transcribed, and translated if appropriate. Although the term “heterologous” is not always used herein in reference to polynucleotides, reference to a polynucleotide even in the absence of the modifier “heterologous” is intended to include heterologous polynucleotides in spite of the omission.

The terms “polypeptide,” “peptide,” and “protein” are used interchangeably herein to refer to polymers of amino acids of any length. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of the intended functionality. The terms also encompass a modified amino acid polymer; for example, disulfide bond formation, glycosylation, lipidation, phosphorylation, methylation, carboxylation, deamidation, acetylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, retaining the desired biochemical function of the intact protein.

The phrases “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism may correlate.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 110%, 125%, 150%, 175%, or 200% or more of the transduction or tropism, respectively, of the control). Suitable controls will depend on a variety of factors including the desired tropism profile. Similarly, it can be determined if a capsid and/or virus “does not efficiently transduce” or “does not have efficient tropism” for a target tissue, or similar terms, by reference to a suitable control.

### V. DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As noted above, provided are compositions and methods for verifying the introduction of a CRISPR/Cas guide RNA into a target cell of interest. In general, a subject method of verifying the introduction of a CRISPR/Cas guide RNA into a target cell of interest includes introducing a nucleic acid encoding a CRISPR/Cas guide RNA into the target cell of interest, extracting DNA (e.g., episomal DNA when non-integrating delivery methods such as AAV or perhaps nanoparticle plasmid delivery are used) from the target cell, and performing an amplification reaction with a primer pair to determine whether the nucleic acid encoding a CRISPR/Cas guide RNA had actually entered the cell.

### Target Cells

A target cell of interest can be any convenient cell type from any convenient organism. For example, the target cell can be a cell cultured in vitro (e.g., an immortalized cell line), a cell ex vivo (a cell extracted from a biological tissue—passaged less than 10 times), or a cell in vivo. The cell can any cell that can to which a subject nucleic acid can be delivered (e.g., a cell that can be infected by AAV, lentivirus, etc. depending on the delivery method employed) —e.g., a rodent cell (e.g., rat, mouse), a human cell, a non-human primate cell (e.g., monkey), a canine cell, a bovine cell, etc. In some cases, the target cell is human cell in vitro. In some cases, the target cell is a rodent cell (e.g., mouse, rat) in vitro. In some cases, the target cell is a non-human primate cell in vitro. The target cell can also be any convenient cell type (from any convenient tissue) to which a subject nucleic acid can be delivered (e.g., that can be infected by AAV, lentivirus etc., depending on the delivery method employed) —e.g., a neuron, a brain cell, a non-neuronal brain cell (e.g., glial cell), a heart cell, a cardiomyocyte, a kidney cell, a liver cell (hepatocyte), a lung cell, a pancreas cell (e.g., beta cell, alpha cell, delta cell, gamma cell), a retinal cell, skeletal muscle cell, a cancer cell, an immortalized cell, a skin cell, and the like. In some cases, the target cell is a non-dividing cell. In some cases, the target cell is a neuron. In some cases, the target cell is a brain cell. In some cases, the target cell is a non-neuronal brain cell, such as a glial cell.

### CRIPSR/Cas Guide RNA and Effector Proteins

### Guide RNA

The nucleic acid introduced into a target cell encodes a CRISPR/Cas guide RNA. Guide RNAs that form a complex with a CRISPR-Cas effector polypeptide are well known in the art. A guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” of a guide RNA.

In many instances, the targeting segment and the protein-binding segment are heterologous to one another. The first segment (targeting segment) of a guide RNA includes a nucleotide sequence (a “guide sequence”) that is complementary to (and therefore hybridizes with) a specific sequence (a target site) within a target nucleic acid (e.g., a target DNA). The protein-binding segment (or “protein-binding sequence”) interacts with (binds to) a CRISPR/Cas effector polypeptide. The protein-binding segment of a guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., a targeted sequence of a target DNA) determined by base-pairing complementarity between the guide RNA (the guide sequence of the guide RNA) and the target nucleic acid.

A guide RNA and a CRISPR/Cas effector polypeptide form a complex (e.g., bind via non-covalent interactions). The guide RNA provides target specificity to the complex by including a targeting segment, which includes a guide sequence (a nucleotide sequence that is complementary to a sequence of a target nucleic acid). The CRISPR/Cas effector polypeptide of the complex provides the site-specific activity (e.g., cleavage activity or an activity provided by the CRISPR/Cas effector polypeptide when the CRISPR/Cas effector polypeptide is a CRISPR/Cas effector polypeptide fusion polypeptide, i.e., has a fusion partner). In other words, the CRISPR/Cas effector polypeptide is guided to a target nucleic acid sequence (e.g. a target sequence in a target DNA) by virtue of its association with the guide RNA.

The term “CRISPR/Cas guide RNA” is generic to a guide RNA in general. When a particular type of guide RNA is intended, e.g., one that interacts with a particular CRISPR/Cas effector polypeptide (e.g., Cas9), then the effector polypeptide's name is used in the guide RNA name—e.g., a “CRISPR/Cas9 guide RNA” is a CRISPR/Cas guide RNA that forms a complex with (and functions with) Cas9.

The “guide sequence” also referred to as the “targeting sequence” of a guide RNA can be modified so that the guide RNA can target a CRISPR/Cas effector polypeptide to any desired sequence of a target DNA, with the exception that the protospacer adjacent motif (PAM) sequence (present on the target DNA) should be taken into account when targeting double stranded DNA.

In some cases, a guide RNA includes two separate nucleic acid molecules and is referred to herein as a “dual guide RNA”, a “double-molecule guide RNA”, or a “two-molecule guide RNA” a “dual guide RNA”, or a “dgRNA.” In some cases, a guide RNA is a single-molecule RNA and is referred to as a “single guide RNA”, or simply “sgRNA.” In the present disclosure, the guide RNA of a subject nucleic acid (e.g., AAV nucleic acid) is an sgRNA.

The targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

The guide sequence of a guide RNA can have a length of from 15 nt to 30 nt (e.g., 15 to 25 nt, 15 to 24 nt, 15 to 23 nt, 15 to 22 nt, 15 to 21 nt, 15 to 20 nt, 15 to 19 nt, 15 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt. In some cases, the guide sequence has a length of from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt).

A guide RNA present in a subject nucleic acid will usually be operably linked to a promoter-usually an RNA polymerase III (pol III) promoter such as a U6 or H1 promoter. In some cases in which the promoter is part of the inversion cassette or the guide RNA is part of the inversion cassette, the promoter will not be operably linked to the guide RNA until after inversion.

### Effector Polypeptide

In class 2 CRISPR systems, the functions of the effector complex (e.g., the cleavage of target DNA) are carried out by a single protein (which can be referred to as a CRISPR/Cas effector protein) —where the natural protein is an endonuclease (e.g., see Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; and Shmakov et al., Nat Rev Microbiol. 2017 March; 15(3):169-182: “Diversity and evolution of class 2 CRISPR-Cas systems”). As such, the term “class 2 CRISPR/Cas protein” or “CRISPR/Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex. In some cases, a subject CRISPR/Cas effector polypeptide is Cas9. Variants of the effectors polypeptides (relative to wild type sequences) are also encompassed as long as a cognate guide RNA can form a complex the effector and together they target a target sequence of a target DNA—determined by the guide sequence of the guide RNA (taking PAM into account). In some cases, the effector is a wild type protein that has DNA cleavage activity. In some cases, the effector protein is a mutant protein that has reduced activity (e.g., is a nickase or a catalytically inactive, i.e., dead version of the protein). In some cases, as is known in the art, a CRISPR/Cas effector protein targets RNA rather that DNA.

In some cases, the CRISPR-Cas effector polypeptide (i.e., CRISPR/Cas effector protein) is a type II CRISPR-Cas effector polypeptide. In some cases, the type II CRISPR-Cas effector polypeptide is a Cas9 polypeptide, e.g., Staphylococcus aureus Cas9 (SaCas9), Streptococcus pyogenes Cas9 (SpCas9), etc. In some cases, the CRISPR-Cas effector polypeptide is a variant of a wild-type SpCas9 and comprises one or more of the following substitutions: A61R, L1111R, A1322R, D1135L, S1136W, G1218K, E1219Q, N1317R, R1333P, R1335A, and T1337R. In some cases, the CRISPR-Cas effector polypeptide is an SpG polypeptide or a SpRY polypeptide; see, e.g., Walton et al. (2020) Science 368:290, and WO 2019/051097. For example, a suitable CRISPR-Cas effector polypeptide is an SpCas9 polypeptide includes D1135V, R1135Q, and T1137R substitutions, relative to wild-type SpCas9. As another example, a suitable CRISPR-Cas effector polypeptide is an SpCas9 polypeptide includes D1135V, R1335Q, T1337R, and G1218R substitutions, relative to wild-type SpCas9. As another example, a suitable CRISPR-Cas effector polypeptide is an SpCas9 polypeptide includes D1135L, S1136W, G1218K, E1219Q, R1335A, and T1337R substitutions, relative to wild-type SpCas9. As another example, a suitable CRISPR-Cas effector polypeptide is an SpCas9 polypeptide includes L1111R, A1322R, D1135L, S1136W, G1218K, E1219Q, R1335A, and T1337R substitutions, relative to wild-type SpCas9. As another example, a suitable CRISPR-Cas effector polypeptide is an SpCas9 polypeptide includes A61R, L1111R, A1322R, D1135L, S1136W, G1218K, E1219Q, N1317R, R1333P, R1335A, and T1337R substitutions, relative to wild-type SpCas9.

In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. In some cases, the CRISPR-Cas effector polypeptide is a Cas7-11 polypeptide; see, e.g., Özcan et al. (2021) Nature 597:720. In some cases, the CRISPR-Cas effector polypeptide is a CRISPRi polypeptide; see, e.g., Qi et al. (2013) Cell 152:1173; and Jensen et al. (2021) Genome Research doi:10.1101/gr.275607.121. In some cases, the CRISPR-Cas effector polypeptide is a CRISPRa polypeptide; see, e.g., Jensen et al. (2021) Genome Research doi:10.1101/gr.275607.121; and Breinig et al. (2019) Nature Methods 16:51. In some cases, the CRISPR-Cas effector polypeptide is a CRISPRoff polypeptide. See, e.g., Nuñez et al. (2021) Cell 184:2503. In some cases, the CRISPR-Cas effector polypeptide is a nickase. In some cases, the CRISPR-Cas effector polypeptide exhibits reduced catalytic activity compared to a wild-type CRISPR-Cas effector polypeptide.

As noted above, in some cases, a CRISPR-Cas effector polypeptide is a Type VI CRISPR-Cas effector polypeptide, such as a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide. As noted above, in some cases, a CRISPR-Cas effector polypeptide is a Type III CRISPR-Cas effector polypeptide. A suitable Type III CRISPR-Cas effector polypeptide is a Cas7-11 polypeptide (see, e.g., Özcan et al. (2021) Nature 597:710). In some cases, a CRISPR-Cas effector polypeptide is an RNA-binding CRISPR-Cas effector polypeptide. For example, in some cases, the CRISPR-Cas effector polypeptide is an RFx Cas13d polypeptide. As another example, in some cases, the CRISPR-Cas effector polypeptide is a dRfxCas13d polypeptide. As another example, in some cases, the CRISPR-Cas effector polypeptide is a DjCas 13d polypeptide. As another example, in some cases, the CRISPR-Cas effector polypeptide is a PspCas13b polypeptide, e.g., Prevotella sp. P5-125 Cas13b. As another example, in some cases, the CRISPR-Cas effector polypeptide is a dPspCas 13b polypeptide In some cases, a Cas13 polypeptide (e.g., a Cas13a polypeptide, a Cas13b polypeptide, a Cas13c polypeptide, a Cas13d polypeptide, a Cas13e polypeptide, a Cas13f polypeptide, a Cas13X polypeptide, or a Cas13Y polypeptide) comprises two Higher Eukaryotes and Prokaryotes Nucleotide-binding (HEPN) domains, each comprising a HEPN motif, where each HEPN motif is RXXXXH, RXXXXXH, or RXXXXXXH, where X is any amino acid.

In some cases, the CRISPR-Cas effector polypeptide is a variant that exhibits reduced catalytic activity compared to a wild-type CRISPR-Cas effector polypeptide. For example, where the CRISPR-Cas effector polypeptide (e.g., a Cas13 polypeptide) comprises a HEPN domain, the CRISPR-Cas effector polypeptide can comprise a substitution of the Arg and/or the His in the HEPN motif, where such a mutation reduces the catalytic activity of the CRISPR-Cas effector polypeptide. In some cases, the RNA-binding CRISPR-Cas effector polypeptide binds, but does not cleave, a target RNA. In some cases, a CRISPR-Cas effector polypeptide is a variant that, when complexed with a guide nucleic acid, binds but does not cleave a target DNA.

As another example, in some cases, the CRISPR-Cas effector polypeptide is a Cas7-11 polypeptide (e.g., a DiCas7-11 polypeptide). See, e.g., Özcan et al. (2021) Nature 597:720. In some cases, the Cas7-11 polypeptide is a variant that exhibits reduced catalytic activity compared to a wild-type Cas7-11 effector polypeptide. For example, in some cases, the variant Cas7-11 polypeptide comprises a substitution of one or more of D177, D429, D654, D758, E959, and D998. For example, a variant Cas7-11 can have an Asp at position 177, an Ala at position 429, an Ala at position 654, an Asp at position 758, a Glu at position E959, and an Asp at position 998. In some cases, a variant Cas7-11 comprises a D429A substitution.

In some cases, an RNA-guided endonuclease (i.e., a CRISPR/Cas effector protein) is a fusion protein that is fused to a heterologous polypeptide (also referred to as a “fusion partner”). In some cases, an RNA-guided endonuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.). In some embodiments, an RNA-guided endonuclease is fused to an amino acid sequence (a fusion partner) that provides a tag (i.e., the fusion partner is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some cases, the fusion partner can provide for increased or decreased stability (i.e., the fusion partner can be a stability control peptide, e.g., a degron, which in some cases is controllable (e.g., a temperature sensitive or drug controllable degron sequence).

An RNA-guided endonuclease (e.g., a Cas9 protein) can have multiple (1 or more, 2 or more, 3 or more, etc.) fusion partners in any combination of the above. As an illustrative example, an RNA-guided endonuclease (e.g., a Cas9 protein) can have a fusion partner that provides for tagging (e.g., GFP), and can also have a subcellular localization sequence (e.g., one or more NLSs). In some cases, such a fusion protein might also have a tag for ease of tracking and/or purification (e.g., a histidine tag, e.g., a 6×His tag; a hemagglutinin (HA) tag; a FLAG tag; a Myc tag; and the like). As another illustrative example, an RNA-guided endonuclease (e.g., a Cas9 protein) can have one or more NLSs (e.g., two or more, three or more, four or more, five or more, 1, 2, 3, 4, or 5 NLSs). In some cases a fusion partner (or multiple fusion partners, e.g., 1, 2, 3, 4, or 5 NLSs) (e.g., an NLS, a tag, a fusion partner providing an activity, etc.) is located at or near the C-terminus of the RNA-guided endonuclease (e.g., Cas9 protein). In some cases a fusion partner (or multiple fusion partners, e.g., 1, 2, 3, 4, or 5 NLSs) (e.g., an NLS, a tag, a fusion partner providing an activity, etc.) is located at the N-terminus of the RNA-guided endonuclease (e.g., Cas9 protein). In some cases the genome editing nuclease (e.g., Cas9 protein) has a fusion partner (or multiple fusion partners, e.g., 1, 2, 3, 4, or 5 NLSs) (e.g., an NLS, a tag, a fusion partner providing an activity, etc.) at both the N-terminus and C-terminus.

In some cases, a fusion partner can modulate transcription (e.g., inhibit transcription, increase transcription) of a target DNA. For example, in some cases the fusion partner is a protein (or a domain from a protein) that inhibits transcription (e.g., a transcription repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like). In many such cases (where the effector protein is fused to a transcription activator or repressor), the effector protein is a catalytically ‘dead’ mutant version of the effector (e.g., dCas9).

In some cases, the fusion partner (heterologous polypeptide) is a reverse transcriptase. In some cases, the fusion partner is a base editor. In some cases, the fusion partner (heterologous polypeptide) is a deaminase.

In some cases, a fusion partner has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In some cases, a fusion partner has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

As noted above, in some cases, the CRISPR/Cas effector protein that is fused to a fusion partner is an enzymatically inactivated protein (a ‘dead’ protein—e.g., dCas9).

CRISPRi: Examples of transcription repressors that can be fused to a CRISPR/Cas effector protein (e.g., Cas9 such as dCas9) include, but are not limited to: Krüppel-associated box (KRAB), SID domain, MeCP2, KRAB-MeCP2, KOX1-KRAB-MeCP2, KRAB-ZIM3, and SALL1-SDS3.

CRISPRa: Examples of transcription activators that can be fused to a CRISPR/Cas effector protein (e.g., Cas9 such as dCas9) include, but are not limited to: VP64, VPR, Suntag, and Synergistic activation mediator (SAM), SunTag-p65-HSF1, and CBP.

In some cases, the CRISPR/Cas effector protein is not fused to a heterologous protein that provides for transcription modulation or protein modification etc., and instead is an active nuclease that cleaves target DNA (in some cases it will cleave one strand, i.e., has nickase activity, and in some cases it will cleave both strands of DNA). If the CRISPR/Cas effector protein causes a double strand break in the target DNA, the cell machinery will repair the damage, and often this will result in mutations (e.g., insertions, deletions, substitutions). When paired with guide RNAs, e.g., from a library, such a protein can be used to generate DNA edits at gene targets as opposed to just increasing or decreasing expression as with CRISPRa and CRISPRi.

### Delivery

As noted herein, in the subject methods, a subject nucleic acid (one that encodes a guide RNA and also includes a probe sequence, e.g., a spacer sequence) can be introduced into a target cell (i.e., delivered to a target cell) (e.g., administered as a guide RNA library to a subject). In some embodiments a recombinase and/or CRISPR/Cas effector protein is also delivered to a target cell (in some cases as DNA, in some cases as RNA, and in some cases as protein).

As would be readily understood by one of ordinary skill in the art, subject nucleic acids (e.g., vectors) and proteins can be delivered to cells using any convenient method.

A “vector” or “expression vector” is a replicon, such as plasmid, phage, virus, or cosmid, to which another DNA segment, e.g., an insert of interest such as a sequence encoding a guide RNA, may be attached so as to bring about the replication and/or expression of the attached segment in a cell. An “expression cassette” comprises a DNA sequence (coding or non-coding) operably linked to a promoter. In some cases a subject vector is a viral vector (e.g., AAV, lentivirus, adenovirus).

Vectors may be provided directly to a target host cell (target cell). In other words, the cells are contacted with vectors comprising the subject nucleic acids (e.g., recombinant expression vectors) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors (e.g., adeno-associated virus (AAV)).

In some embodiments, a subject vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Methods of introducing nucleic acids and/or proteins into a target cell are known in the art, and any convenient method can be used. Suitable methods include, e.g., viral infection (e.g., AAV, adenovirus, lentiviral), transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9), and the like.

A subject protein can be introduced into a cell (provided to the cell) by any convenient method; such methods are known to those of ordinary skill in the art. As an illustrative example, a subject protein can be injected directly into a cell. As another example, a subject protein can be introduced into a cell via nucleofection; via a protein transduction domain (PTD) conjugated to the protein, etc.

In some cases, a subject nucleic acid and/or protein is delivered to a target cell in a particle, or associated with a particle. In some cases, a subject protein is delivered with a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5).

A subject nucleic acid and/or protein may be delivered using particles or lipid envelopes. For example, a biodegradable core-shell structured nanoparticle with a poly(β-amino ester) (PBAE) core enveloped by a phospholipid bilayer shell can be used. In some cases, particles/nanoparticles based on self assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used.

Lipidoid compounds (e.g., as described in U.S. patent application No. 20110293703) are also useful in the administration of polynucleotides, and can be used to deliver a subject protein (or RNA or DNA encoding it). In one aspect, the aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver a subject protein or nucleic acid to a target cell. US Patent Publication No. 20130302401 relates to a class of poly(beta-amino alcohols) (PBAAs) that has been prepared using combinatorial polymerization.

Sugar-based particles may be used, for example GalNAc, as described with reference to WO2014118272 (incorporated herein by reference) and Nair, J K et al., 2014, Journal of the American Chemical Society 136 (49), 16958-16961) can be used to deliver a subject protein or nucleic acid to a target cell.

In some cases, lipid nanoparticles (LNPs) are used to deliver a subject nucleic acid and/or protein to a target cell. Preparation of LNPs will be known to one of ordinary skill in the art, e.g., as described in, e.g., Rosin et al. (2011) Molecular Therapy 19:1286-2200). As non-limiting examples, cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLink-DMA), 1,2-dilinoleyl-4-(2-dimethylaminoethyl)-[1,3]-dioxolane (DLinKC2-DMA), (3-O-[2″-(methoxypolyethyleneglycol 2000) succinoyl]-1,2-dimyristoyl-sn-glycol (PEG-S-DMG), and R-3-[(.omega.-methoxy-poly(ethylene glycol) 2000) carbamoyl]-1,2-dimyristyloxlpropyl-3-amine (PEG-C-DOMG) may be used. A nucleic acid may be encapsulated in LNPs containing DLinDAP, DLinDMA, DLink-DMA, and DLinKC2-DMA (cationic lipid:DSPC:CHOL:PEGS-DMG or PEG-C-DOMG at 40:10:40:10 molar ratios). In some cases, SP-DiOC18 (e.g., 0.2% SP-DiOC18) is incorporated.

Spherical Nucleic Acid (SNA™) constructs and other nanoparticles (particularly gold nanoparticles) can be used to deliver a subject nucleic acid and/or protein to a target cell. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495: S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

Nanoparticles suitable for use in delivering a subject protein or nucleic acid to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for use in delivering a subject protein or nucleic acid to a target cell. A prototype nanoparticle of semi-solid nature is the liposome.

In some cases, an exosome is used to deliver a subject nucleic acid and/or protein to a target cell. Exosomes are endogenous nano-vesicles that transport RNAs and proteins, and which can deliver RNA to the brain and other target organs.

In some cases, a liposome is used to deliver a subject nucleic acid and/or protein to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver a subject nucleic acid and/or protein to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol) 2000) carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver a subject nucleic acid and/or protein to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a subject protein or nucleic acid to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a subject protein or nucleic acid using a spontaneous vesicle formation procedure. The component molar ratio may be about 50/10/38.5/1.5 (DLin-KC2-DMA or C12-200/disteroylphosphatidyl choline/cholesterol/PEG-DMG).

A subject nucleic acid and/or protein may be delivered encapsulated in PLGA microspheres such as that further described in US published applications 20130252281 and 20130245107 and 20130244279.

Supercharged proteins can be used to deliver a subject nucleic acid and/or protein to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver a subject nucleic acid and/or protein to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

An implantable device can be used to deliver a subject nucleic acid and/or protein to a target cell (e.g., a target cell in vivo, where the target cell is a target cell in circulation, a target cell in a tissue, a target cell in an organ, etc.). An implantable device suitable for use in delivering a subject protein or nucleic acid to a target cell (e.g., a target cell in vivo, where the target cell is a target cell in circulation, a target cell in a tissue, a target cell in an organ, etc.) can include a container (e.g., a reservoir, a matrix, etc.) that comprises the subject protein or nucleic acid.

In some cases, desirable delivery systems provide for roughly uniform distribution and have controllable rates of release of their components (e.g., vectors, proteins, nucleic acids, drugs etc.). A variety of different media are described below that are useful in creating composition delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated include materials such as gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

In some cases, subject compositions are delivered as therapeutic agents (e.g., administered to a subject in vivo).

In some cases, a carrier/medium can include a microparticle. Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids—in any combination thereof.

In some cases, a carrier/medium can include a liposome that is capable of attaching and releasing therapeutic agents (e.g., the subject nucleic acids and/or proteins). Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life.

In some embodiments, a cationic or anionic liposome is used as part of a subject composition or method, or liposomes having neutral lipids can also be used. Cationic liposomes can include negatively-charged materials by mixing the materials and fatty acid liposomal components and allowing them to charge-associate. The choice of a cationic or anionic liposome depends upon the desired pH of the final liposome mixture. Examples of cationic liposomes include but are not limited to: lipofectin, lipofectamine, and lipofectace.

### AAV

In some embodiments, introducing the nucleic acid encoding a CRISPR/Cas guide RNA to a target cell is performed using an AAV that delivers the nucleic acid to the cell. Using an AAV to deliver a nucleic acid is also referred to herein as “contacting” a target cell with an AAV. “AAV” is an abbreviation for adeno-associated virus, and may be used to refer to the virus itself or derivatives thereof, and can be used to refer to a viral vector system for delivery of nucleic acids. The term covers all subtypes and both naturally occurring as well as recombinant and variant forms, except where specified otherwise.

The term “AAV” includes any convenient AAV type (e.g., capsid type), including variant types, e.g., AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV 9_hu14, AAV-DJ (also referred to herein as simply “DJ”), AAV-LK03 (also referred to herein as simply “LK03”), AAV_PHP.eB, avian AAV, bovine AAV, canine AAV, equine AAV, primate AAV, non-primate AAV, and ovine AAV. “Primate AAV” refers to AAV capable of infecting primates, “non-primate AAV” refers to AAV capable of infecting non-primate mammals, “bovine AAV” refers to AAV capable of infecting bovine mammals, etc. Any convenient AAV capsid can be used and it will vary depending on the targeted cell/tissue/organ type. As one illustrative specific example, in some cases, transduction of neurons through intravenous delivery is desired and an AAV-PHP.eB capsid is used. The choice of capsid will likely depend on the target cell type/tissue as well as the desired delivery route, and one of ordinary skill in the art will be able to select an appropriate capsid as is convenient.

As would be clear to one of ordinary skill in the art, it is to be understood that the term “AAV vector” can be used to refer to the delivery system as a whole (e.g., a virion or population of virions), and can also be used to refer to a nucleic acid intended to be delivered to a target cell (i.e., the cargo, also referred to as the “AAV transfer vector”) —i.e., one that includes a nucleotide sequence of interest (e.g., one that includes a subject inversion cassette and that encodes a CRISPR/Cas guide RNA) —flanked by AAV inverted terminal repeat sequences (ITRs).

An “AAV virion” or “AAV virus” or “AAV viral particle” or “AAV vector particle” refers to a viral particle composed of at least one AAV capsid polypeptide (variant AAV capsid polypeptides and/or non-variant parent capsid polypeptides) and an encapsidated polynucleotide AAV transfer vector. Such a particle can be referred to as an “AAV vector particle” or simply an “AAV vector”. Thus, production of AAV virion or AAV particle necessarily includes production of AAV vector as such a vector is contained within an AAV virion or AAV particle.

“Packaging” refers to a series of intracellular events resulting in the assembly of AAV virions or AAV particles which encapsidate a nucleic acid sequence of interest. Packaging can refer to encapsidation of a nucleic acid sequence of interest into a capsid comprising AAV capsid polypeptides.

AAV “rep” and “cap” genes refer to polynucleotide sequences encoding replication and encapsidation proteins of adeno-associated virus (AAV). AAV rep (replication) and cap (capsid) are referred to herein as AAV “packaging genes.”

A “helper virus” for AAV refers to a virus allowing AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used as a helper virus. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome allowing AAV replication and packaging (in conjunction with other requirements for replication and packaging described herein). As “helper virus function” may be provided in a number of ways, including by providing helper virus or providing, for example, polynucleotide sequences encoding the requisite function(s) to a producer cell in trans.

An “infectious” virion, virus or viral particle is one comprising a polynucleotide component deliverable into a cell tropic for the viral species. The term does not necessarily imply any replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, enter a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

A “replication-competent” virion or virus (e.g. a replication-competent AAV) refers to an infectious phenotypically wild-type virus, and is replicable in an infected cell (i.e. in the presence of a helper virus or helper virus functions). In the case of AAV, replication competence generally requires the presence of functional AAV packaging genes. In some embodiments, AAV vectors, as described herein, lack of one or more AAV packaging genes and are replication-incompetent in mammalian cells (especially in human cells). In some embodiments, AAV vectors lack any AAV packaging gene sequences, minimizing the possibility of generating replication competent AAV by recombination between AAV packaging genes and an incoming AAV vector. In many embodiments, AAV vector preparations as described herein are those containing few if any replication competent AAV (rcAAV, also referred to as RCA) (e.g., less than about 1 rcAAV per 10\hat{ }2 AAV particles, less than about 1 rcAAV per 10\hat{ }4 AAV particles, less than about 1 rcAAV per 10\hat{ }8 AAV particles, less than about 1 rcAAV per 10\hat{ }12 AAV particles, or no rcAAV).

### Recombinase Protein and Target Sites

A recombinase, expressed by the target cell of interest, facilitates the verification of introduction of a CRISPR/Cas guide RNA into the cell. The target cell of interest expresses a recombinase protein that inverts a probe sequence (e.g., a spacer sequence, the sgRNA promoter sequence, the sgRNA sequences, etc. as described elsewhere herein) on the subject nucleic acid (the nucleic acid encoding the CRISPR/Cas guide RNA). The probe sequence (e.g., spacer sequence) is therefore only inverted in cells expressing the recombinase protein (i.e., target cells of interest). As discussed elsewhere herein, in some cases the recombinase generates a neo-sequence as a result of excision/deletion instead of inversion.

A recombinase protein can be any convenient recombinase that catalyzes inversion of a spacer sequence flanked by target sites for the recombinase. For example, in some cases, the recombinase is a tyrosine-type site-specific recombinase (Y-SSR; also referred to in the art as T-SSR). Y-SSRs include, but are not limited to Cre, Flp, R recombinase, and Pin1 recombinase. In some cases, a subject recombinase protein is a Cre recombinase. In some cases, a subject recombinase protein is a Flp recombinase. In some cases, a subject recombinase protein is an R recombinase. In some cases, a subject recombinase protein is a Pin1 recombinase. In some cases, the recombinase protein is selected from the group consisting of Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spolVCA, PhiMR11, SCCmec, TndX, XerC, XerD, XisA, Hin, Cin, mrpA, beta, PhiFC1, Fre, Clp, sTre, FimE, and HbiF.

Recombinases can be classified into two distinct families: serine recombinases and tyrosine recombinases, based on distinct biochemical properties. Serine recombinases and tyrosine recombinases can further be divided into bidirectional recombinases and unidirectional recombinases. Examples of bidirectional serine recombinases include, without limitation, β-six, CinH, ParA and γδ; and examples of unidirectional serine recombinases include, without limitation, Bxb1, ΦC31 (phiC31), TP901, A118, TGI, phiBT1, R4, cpRV1, cpFC1, MRU, A118, U153 and gp29. Examples of bidirectional tyrosine recombinases (YSSRs) include, without limitation, Cre, FLP, Pin1, and R; and unidirectional tyrosine recombinases include, without limitation, Lambda, HK1O1, HK022 and pSAM2.

Cognate target sites for the recombinase of choice will be known and readily available to one of ordinary skill in the art (see, e.g., Albert et al., 1995 Plant J., 7, 649-659; Zhang et al., Nucleic Acids Res. 2002 Sep. 1; 30(17):e90; and Schmitt et al., Nat Commun. 2022 Dec. 27; 13(1):7966). Examples of target sites include, but are not limited to: Lox (for Cre) (e.g., LoxP and mutant Lox sites such as Lox71 and Lox66, which are converted during inversion to Lox72 and LoxP, respectively) and FRT (for Flip), LoxP, loxN, lox511, lox5171, lox2272, LoxM2, LoxM3, LoxM7, LoxM11, lox71, lox66, FRT, FRT11, FRT71, attp, att, rox, SloxM1, VloxP, vox, B3RT, KDRT, F3 (FRT), F14 (FRT), attB/P, F5, F13, Vlox2272, Slox2272, SloxP, RSRT, and B2RT.

In some cases, the recombinase is a unidirectional recombinase. In some cases, the target sites are selected such that there is a bias toward unidirectional recombination (e.g., use of Lox71 and Lox66 with Cre recombinase).

### Expression of the Recombinase and the CRISPR/Cas Effector Protein

Because a subject functional screen depends on the presence of two proteins (a CRISPR/Cas effector protein and the recombinase protein for inversion of sgRNAs constructs, which is a prerequisite for their amplification during screen analysis), only cells of interest that express both proteins will be evaluated.

In some cases, the target cells of interest (those expressing a recombinase protein such as Cre) are a subpopulation of the target cells that are infected with the library (e.g., AAV library, lentiviral library and the like). For example, in some cases the population of target cells might be brain cells, but the only brain cells of interest are neurons. In such cases, the recombinase protein (e.g., a YSSR such as Cre, or a serin recombinase such as phiC31) can be expressed from a tissue or cell-type specific promoter, e.g., a neuron specific promoter—thus, the amplification reaction will only detect guide RNA sequences present in those recombinase-expressing cells because those are the only cells in which the probe sequence (e.g., spacer sequence) was inverted and therefore the only cells for which the two primers will form a primer pair. In such cases, the CRISPR/Cas effector protein should be expressed from the same promoter as the recombinase protein, or from a promoter that expresses in the same cells plus additional cells, e.g., a tissue non-specific promoter such as a ubiquitous promoter (e.g., EF1a, CMV, b-actin, and the like).

In some cases, the recombinase protein is already expressed in the target cells of interest before the library is introduced, e.g., a subject such as a recombinase-expressing mouse can be used (e.g., a Cre-expressing mouse). The recombinase can be expressed from any convenient promoter, e.g., tissue-specific promoter (such as a neuron-specific promoter), a temporally controlled promoter, an inducible promoter, a ubiquitous promoter (e.g., EF1a, beta-actin, CMV), and the like. Likewise, the recombinase can be introduced or (or its expressed induced) just before or along with the library. In some cases, the recombinase protein is introduced via AAV, e.g., a second AAV (in addition to the guide RNA library AAVs) can be used.

Similarly, in some cases, the CRISPR/Cas effector protein (e.g., a CRISPRi protein or a CRISPRa protein, both of which are describe elsewhere herein) is already expressed in the target cells of interest before the library is introduced, e.g., a subject such as a recombinase-expressing mouse can be used (e.g., a Cas9-expressing mouse). The CRISPR/Cas effector protein can be expressed from any convenient promoter, e.g., tissue-specific promoter (such as a neuron-specific promoter), a temporally controlled promoter, an inducible promoter, a ubiquitous promoter (e.g., EF1a, beta-actin, CMV), and the like. Likewise, the CRISPR/Cas effector protein can be introduced or (or its expressed induced) just before or along with the library. In some cases, the CRISPR/Cas effector protein is introduced via AAV, e.g., a second AAV (in addition to the guide RNA library AAVs) can be used.

As such, in some embodiments, a subject method includes introducing the recombinase protein (e.g., YSSR such as Cre, or a serin recombinase such as phiC31) and/or the CRISPR/Cas effector protein (e.g., Cas9) to the target cell. In some cases, target cells are contacted with an AAV that delivers a nucleic acid encoding the recombinase protein (e.g., YSSR such as Cre, or a serin recombinase such as phiC31) and/or the CRISPR/Cas effector protein (e.g., a Cas9-based CRISPRi or CRISPRa).

In some cases, prior to introducing the library, the target cell includes a nucleic acid encoding the recombinase protein and/or the CRISPR/Cas effector protein. In some cases, expression of the recombinase protein is controlled by a tissue specific promoter (e.g., a neuron-specific promoter such as an hSyn1 promoter). In some cases, expression of the CRISPR/Cas effector protein is controlled by a tissue specific promoter (e.g., a neuron-specific promoter such as an hSyn1 promoter). In some cases, expression of the recombinase protein is controlled by a ubiquitous promoter. In some cases, expression of the CRISPR/Cas effector protein is controlled by a ubiquitous promoter.

### Probe Sequence

The subject methods include an inversion of a probe sequence (also referred to herein as an “inverting probe sequence” or simply “probe”) that is part of an inversion cassette. Thus, in addition to a nucleotide sequence encoding a CRISPR/Cas guide RNA (i.e., the nucleic acid encodes a CRISPR/Cas guide RNA), a subject nucleic acid also includes an “inversion cassette” (also referred to as a “probe cassette”). An inversion cassette includes a probe sequence (e.g., spacer sequence) flanked by target sites for a recombinase protein (e.g., Cre, Flp, phiC31 and the like) —and in the subject methods, the recombinase protein will be present in the target cells. The probe sequence is inverted by the recombinase because it is flanked by (positioned between) the target sites. In short, inversion of the probe sequence establishes a record of a target cell of interest having received the guide RNA and having expressed/contained the active recombinase (e.g., Cre). A subject probe sequence can be any convenient sequence that allows for a primer (of a primer pair-see more details below) to hybridize and thereby distinguish the orientation of the sequence. The probe sequence thereby provides for an enzyme-dependent sequence change (inversion) that allows for priming of an amplification reaction to detect only those nucleic acids that underwent the inversion and therefore were present in target cells of interest (i.e., target cells that contained the recombinase).

In some cases, the probe sequence can include the promoter that controls the expression of the guide RNA (e.g., a pol III promoter such as a U6 promoter, an H1 promoter, and the like), in which case the promoter would be in an improper orientation prior to inversion (improper for controlling expression of the guide RNA) such that the guide RNA would only be expressed after inversion. In some cases, the probe sequence can include the CRISPR/Cas guide RNA in an improper orientation relative to its promoter prior to inversion such that the guide RNA would only be expressed by the promoter after inversion. In some cases, the probe sequence can include both the CRISPR/Cas guide RNA and the promoter to which it is operably linked. In some such cases, the guide RNA can be expressed when the probe sequence is in either orientation such that inversion does not alter expression of the guide RNA, but does still generate a new primer pair such that the inversion can be detected using an amplification reaction.

In some cases, the recombinase catalyzes a deletion/excision instead of an inversion, generating a new sequence (a “neo-sequence”) that was not present prior to deletion/excision—and such sequence can then function as a probe sequence for amplification. In other words, in such cases a primer that can hybridize to the neo-sequence can used for detection in the amplification reaction.

In some embodiments, the probe sequence is a “spacer sequence” (also referred to herein as an “inverting spacer sequence” or simply “spacer”). When the probe sequence is a spacer sequence, the inversion cassette can also be referred to as a spacer cassette (a spacer sequence flanked by target sites for the recombinase). A spacer sequence is an additional sequence that is not part of the guide RNA expression cassette (i.e., not part of the CRISPR/Cas guide RNA or the promoter to which it is operably linked). In some embodiments, the probe sequence (e.g., the inverting spacer sequence (“spacer”)) has a length in a range of from 50-2,000 base pairs (bp) (e.g., 50-1,500, 50-1,000, 50-800, 50-600, 50-500, 50-400, 50-300, 50-200, 100-2,000, 100-1,500, 100-1,000, 100-800, 100-600, 100-500, 100-400, 100-300, 100-200, 200-2,000, 200-1,500, 200-1,000, 200-800, 200-600, 200-500, 200-400, 200-300, 200-200, 300-2,000, 300-1,500, 300-1,000, 300-800, 300-600, 300-500, or 300-400 bp). In some cases, the probe sequence (e.g., spacer) has a length in a range of from 100-300 bp (e.g., 100-250, 100-200, 150-250, 150-200 bp). In some cases, the probe sequence (e.g., spacer) has a length of about 50, 100, 150, 175, or 200 bp. In some cases, the probe sequence (e.g., spacer) has a length of about 175 bp. In some cases, the probe sequence (e.g., spacer) has a length of from 100-400 base pairs.

An inverting spacer sequence (“spacer”) can be any convenient sequence that allows for a primer (of a primer pair-see more details below) to hybridize and thereby distinguish the orientation of the sequence. In the working example below (Example 1) the spacer sequence used was:

(SEQ ID NO: 1)
ACATGTAAACGATCCTAGGCGTCCATATGATGCATGGCAGCAATT
GGCCATTTGTAGGCAGACGCATCGATCAGTACAGTCGGATAGTCG
AACGATCATTACAGTCAGTCGAATGCAAAGTAGCGAGGCATGACC
TGTGTACTTAAAGCATGACTGGTACTGACACGTCGCATGC

Thus, in some embodiments, the spacer sequence comprises the sequence of SEQ ID NO: 1. The portion of the primer that bound to the spacer sequence used in Example 1 is TGACTGGTACTGACACGTCG (SEQ ID NO: 3) —this sequence is in bold above. Thus, in some cases, the primer that hybridizes to the probe sequence (e.g., the spacer sequence) includes the sequence set forth in SEQ ID NO: 3.

### DNA Extraction

Any convenient method for extracting DNA from a sample of cells can be used, and one of ordinary skill in the art will be able to select from a number of suitable options. In some embodiments, extraction is performed in order to obtain episomes. When using some delivery methods, the subject nucleic acid (which includes the guide RNA encoding sequence and the inversion cassette) is not integrated into the genome of the target cells, but is instead episomal. Therefore, episome DNA extraction can serve as a way to enrich a sample of extracted DNA for episomal nucleic acids. For example, because AAV vectors are usually not integrated once delivered to a target cell, but are instead maintained episomally, episome DNA extraction can serve as a way to enrich a sample of extracted DNA for the AAVs that were introduced into cells as part of a subject method. As such, in some cases, the extracted DNA of a subject method includes episomal DNA. in some cases, the extracted DNA of a subject method is enriched for episomal DNA. in some cases, the only DNA in a sample of extracted DNA of a subject method is episomal DNA.

In some cases, the delivery method employed (e.g., lentiviral delivery) results in integration of the subject nucleic acid into the genome of the target cells. As such, in some cases, DNA extraction is performed in order to obtain genomic DNA. In some cases, DNA extraction is performed in order to obtain genomic and episomal DNA.

Amplification with a Primer Pair

To detect inversion of the probe sequence (e.g., spacer sequence), and thereby detect the introduction of a subject nucleic acid (one the encodes a CRISPR/Cas guide RNA and includes an inversion cassette) into a target cell of interest (i.e., a target cell that expresses the recombinase protein), an amplification reaction is performed using two primers.

As used herein, the term “primer oligonucleotide” (or simply “primer”) refers to a molecule comprising at least one continuous strand of from about 12 to about 100 nucleotides in length and sufficient to permit enzymatic extension during an amplification process such as polymerase chain reaction. Selection of an appropriate length of primer given the reaction conditions, sequence being targeted, etc. will be routine and straightforward for one of ordinary skill in the art. In some cases, the first and second primer, independently, have a length of from 15 to 50 nucleotides (e.g., 15-40, 15-30, 15-25, 15-22, 15-20, 17-50, 17-40, 17-30, 17-25, 17-22, 17-20, 18-50, 18-40, 15-30, 18-25, 18-22, 18-20, 19-50, 19-40, 19-30, 19-25, 19-22, or 19-20 nucleotides in length). In some cases, the first and second primer, independently, have a length of from 18 to 25 nucleotides.

One of the two primers hybridizes to a sequence within the probe sequence (e.g., spacer sequence) such that the two primers do not form a primer pair prior to the inversion (i.e., they hybridize to the same strand of the double stranded target DNA prior to the inversion), but do form a primer pair after the inversion (i.e., they hybridize to opposite strands of the double stranded target DNA after inversion of the probe sequence). A positive result from the amplification reaction—an amplified product of the expected size (or sequence) —indicates that guide RNA entered the cell (because the nucleic acid was exposed to recombinase expressed by the cell).

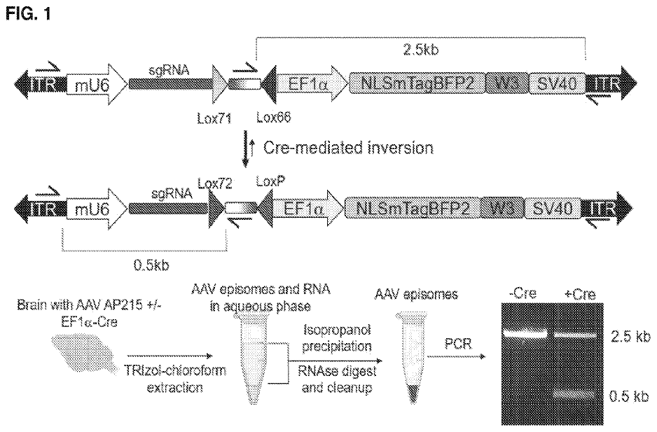

By “primer pair” it is meant a pair of primers that includes one forward primer and one reverse primer, where the two primers hybridize to different strands of a double stranded DNA target such that an amplification reaction would result in amplification of the sequence between the two primers (i.e., the intervening sequence can be amplified). Whether or not two particular primers form a “primer pair” depends on the target molecule to be amplified. For example, for the top double stranded DNA molecule depicted in the schematic illustration of FIG. 1, there are 3 total primers depicted (half-arrows): left, middle, and right. The middle primer does not form a primer pair with the left primer because they hybridize to the same strand of the double stranded DNA target, and therefore an amplification reaction would not amplify the intervening sequence. Instead, the middle primer forms a primer pair with the right primer because an amplification reaction can amplify the sequence that intervenes the two primers. In the schematic example, the intervening sequence is 2.5 kb in length.

On the other hand, for the double stranded DNA molecule depicted just below the top one, the middle primer now forms a primer pair with the left primer because they now hybridize to opposite strands of the double stranded DNA target (the depicted intervening sequence is 0.5 kb in length), and the middle primer no longer forms a primer pair with the right primer because those two primers now hybridize to the same strand of the double stranded DNA target, and an amplification reaction would not amplify the intervening sequence.

As illustrated in FIG. 1. The only difference between the top depicted DNA molecule and the DNA molecule depicted just below is that the lower DNA molecule is the result of a recombinase (e.g., YSSR) catalyzed inversion of the probe sequence (e.g., spacer sequence). In the depicted example, the YSSR is Cre recombinase and the first and second target sites are Lox sites. Thus, if a primer hybridizes to a probe sequence positioned between first and a second target sites for the recombinase (e.g., YSSR), then inversion of the probe sequence catalyzed by the recombinase converts the primer from a forward primer to a reverse primer (or visa versa) —and a primer pair is created or destroyed.

As such, when a subject method includes a step of performing an amplification reaction with a primer pair after a recombinase (e.g., YSSR such as Cre, or a serin recombinase such as phiC31) catalyzes a probe sequence (e.g., spacer sequence) inversion, and one of the primers of the pair hybridizes to the probe sequence (where the probe sequence is positioned between a first and a second target site for the recombinase, then either: (1) the two primers are considered a primer pair after inversion, but not before inversion (e.g., the middle and left primers depicted in FIG. 1); or (2) the two primers are considered a primer pair before inversion, but not after inversion (e.g., the middle and right primers depicted in FIG. 1). The subject methods are generally focused on the scenario in which two primers are considered a primer pair after probe sequence inversion, but not before probe sequence inversion (e.g., the middle and left primers depicted in FIG. 1). In some embodiments in which the recombinase catalyzes excision/deletion instead of inversion, thus generating a neo-sequence, the two primers are not considered a primer pair prior to excision because the neo-sequence targeted by one of the primers is not yet present.

A subject amplification reaction that uses two primers can be any convenient type of amplification, e.g., polymerase chain reaction (PCR).

### Libraries

As noted above, and discussed more below, being able to identify the guide sequence(s) present in the amplified product from the amplification reaction allows for functional screening based on this approach. Thus, also provided are compositions and methods for CRISPR-based functional screening in vivo in non-human subjects. Such methods can be used to identify target sequences (e.g., genes) of interest that play roles in phenotypes such as cell survival, cell death, cell proliferation, etc.

For CRISPR-based in vivo screening methods in non-human subjects, a library of nucleic acids encoding CRISPR/Cas guide RNAs (where the guide RNAs of the members of the library have different guide sequences from one another) are introduced to a population of target cells (e.g., brain cells) . . . . Such a library is sometimes referred to herein as a “guide RNA library.” A library can be introduced using any convenient method (see e.g., the section title “Delivery” above), e.g., via virus such as AAV or lentiviral infection.

The term “library” herein is used to refer to a plurality (i.e., a collection of 2 or more, e.g., 5 or more, 10 or more, 100 or more, 500 or more, 1,000 or more, 2,000 or more, 5,000 or more, 10,000 or more, and the like). As an example, each member of a plurality/library of nucleic acids (e.g., AAV nucleic acids) would include the same inversion cassette, but have different guide sequences. As such, a subject library can be considered a “guide RNA library” or an “AAV vector library” or an “AAV guide RNA vector library”, etc., In cases where a delivery vehicle other than AAV is used, e.g., a lentivirus, similar considerations would apply, e.g., the nucleic acids would be the same, but would have different guide sequences, and therefore a subject library could be considered a “guide RNA library” or a “lentivirus vector library” or a “lentivirus guide RNA vector library.”

Thus, provided are libraries of the above nucleic acid vectors (e.g., AAV vectors, lentiviral vectors, etc), where the encoded CRISPR/Cas guide RNAs of the members of the library have different guide sequences from one another.

### Obtaining a Biological Sample/Cell Sample

In some cases, after introducing a guide RNA library into a population of target cells (e.g., contacting the population of target cells with an AAV guide RNA library), a period of time is allowed to pass (the “wait time”) prior to obtaining the cells for DNA extraction (e.g., episomal DNA). This allows the cells time to develop/exhibit a phenotype due to expression of the guide RNAs. A biological sample (a cell sample) (one that includes the contacted population of target cells) can be collected from the subject in any convenient time frame, e.g., depending on the phenotype of interest. For example, in some cases, a biological sample (a cell sample) (one that includes the contacted population of target cells) is collected from the subject in a range of from 1 day to 2 years (e.g., 1-2 yrs, 6 months-2 yrs, 6 months-1.5 yrs, 6 months to 1 year, 1-12 months, 1-10 months, 1-8 months, 1-6 months, 1-4 months, 1-2 months, 1-14 days, 1-12 days, 1-10 days, 1-7 days, 1-4 days, 1-2 days, 3-14 days, 3-10 days, 3-7 days, 1-8 weeks, 2-7 weeks, 3-6 weeks, about 4 weeks, about 5 weeks, about 6 weeks, about 7 weeks, or about 8 weeks) after introducing the library. In some cases, a biological sample (a cell sample) (one that includes the contacted population of target cells) is collected from the subject in a range of from 1 day to 6 months (e.g., 1-8 weeks, 2-7 weeks, 3-6 weeks, about 4 weeks, about 5 weeks, about 6 weeks, about 7 weeks, or about 8 weeks) after introducing the library.

“Biological Sample” (also referred to herein as a “cell sample”) —The biological sample can be from any tissue or organ (e.g., brain, heart, kidney, liver, lung, pancreas, retina, or skeletal muscle), and in some cases the sample is the whole organ. The term “biological sample” encompasses a variety of sample types obtained from an organism. The term encompasses blood and other liquid samples of biological origin or cells derived therefrom and the progeny thereof. The term encompasses samples that have been manipulated in any way after their procurement, such as by treatment with reagents, solubilization, or enrichment (e.g., via fluorescence-activated cell sorting (FACS), magnetic-activated cell sorting (MACS), and the like). The term encompasses cell supernatants, cell lysates, serum, plasma, biological fluids, and tissue samples (e.g., tissue taken from a site of nucleic acid introduction, e.g., AAV introduction). Once a sample is isolated (i.e., collected), it can be used directly (i.e., DNA can be extracted), or can be frozen or fixed (as long as DNA can still later be extracted and amplified), or maintained in appropriate culture medium for a period of time.

In some cases, the biological sample is a whole organism (e.g., if performing a subject screen using small animals such as fruit flies (e.g., Drosophila) or worms (e.g., C. elegans).

In some cases, the biological sample is stored for a period of time (e.g., frozen or fixed, as long as DNA can still later be extracted and amplified) prior to extraction of DNA.

### Detecting and Measuring

If the sequence encoding the guide sequence of the CRISPR/Cas guide RNA is positioned within the introduced nucleic acid such that it is between the two primers of the primer pair (after inversion of the probe sequence), then the amplification product of the amplification reaction will include the guide sequence. The amplification product can be sequenced (e.g., via sequencing such as high-throughput/next-generation DNA sequencing) to identify the guide sequence. Detecting and measuring can be performed via any convenient method, e.g., a method that includes high-throughput sequencing and quantification of the number of sequence reads for each detect guide sequence.

When using a library (in a screening method), once the DNA is extracted and the amplification reaction performed, the amplification product (a sample of amplified guide sequences) should include guide sequences for the guide RNAs still present in the target cell population. For example, for guide RNAs that lead to cell death over the course of the ‘wait time’, those cells will not be present in the cell population by the time cells are obtained and DNA extracted—and therefore those guide sequences will be absent or present at a lower frequency in the amplification product. Thus, in some embodiments, a subject method includes a step of detecting and measuring quantities of guide sequences present in the amplification product. The result of said detecting and measuring (a ‘test set of measured values’) can then be compared to a control (a ‘control set of measured values’) to determine whether any of the guide sequences are enriched or depleted in the test set relative to the control set. The control set can provide quantities (e.g., quantities of the different guide sequences relative to one another) that were present in library (e.g., library of AAVs, library of lentiviruses, and the like) prior to said contacting. In other words, the amplification product can be assayed (e.g., via high throughput/nextgen sequencing) to determine the relative abundance of the different guide sequences present after contacting the cell population compared to the relative abundance that had been present prior the contacting. Alternatively, the control set can be amplified from cells which did make contact with the guide RNA library, but which lack the CRISPR/Cas effector protein (or lack the ability to activate it). Such a control set may be an organism which lacks the DNA sequences in its genome for creating the CRISPR/Cas effector protein, or an organism whose control of creating the CRISPR/Cas effector protein is activated by a regulatory mechanism, such as mice with a lox-stop-lox-driven CRISPR/Cas effector protein which did not receive the recombinase to activate the effector protein.

The nature of the CRISPR/Cas effector protein will affect the interpretation of the results of such screening. For example, in some cases the CRISPR/Cas effector protein (e.g., Cas9) is fused to a transcription repressor (i.e., CRISPRi) (e.g., KRAB). In such cases, a guide sequence that targets a protein essential for cell survival should lead to decreased expression of the cell survival protein and perhaps cell death, which would result in depletion of that guide sequence from the amplification product. For example, for guide RNAs that would knock down expression of essential genes (e.g., neuronal essential genes), those guide RNAs would be depleted from the library after contacting the target cells relative to the starting library (prior to contact).

On the other hand, in some cases the CRISPR/Cas effector protein (e.g., Cas9) is fused to a transcription activator (i.e., CRISPRa) (e.g., VP64, e.g., using a synergistic activation mediator (SAM) system). In such cases, a guide sequence that targets a protein that is toxic at high levels should lead to increased expression of the toxic protein and perhaps cell death, which would result in depletion of that guide sequence from the amplification product.

Thus, in some embodiments, the CRISPR/Cas effector protein (e.g., Cas9) is fused to a transcription repressor (i.e., CRISPRi) (e.g., KRAB). In some embodiments, the CRISPR/Cas effector protein (e.g., Cas9) is fused to a transcription activator (i.e., CRISPRa) (e.g., VP64, e.g., using a synergistic activation mediator (SAM) system).

When using high-throughput/nextgen sequencing, the number of sequence reads for each unique guide sequence can be used to determine the relative abundance of that guide sequence (e.g., relative to other guide sequences present in the amplification product). The number of sequence reads can be absolute (e.g., an actual number of molecules per volume of sample) or can be relative (e.g., relative to a pre-determined control, such as a spiked guide RNA sequence or a guide sequence that targets a particular control gene).

Additional screens using the subject methods are also envisioned herein. The following are non-limiting examples: (1) screens based on selection of cell populations with a phenotype of interest by fluorescence-activated cell sorting (FACS) or magnetic-activated cell sorting (MACS), where guide RNA frequencies are compared between cells selected vs. unselected by MACS or FACS, or cells with different fluorescent parameters (reporting on phenotypes of interest) separated by FACS; (2) screens based on migration of a cell type from one tissue area to another, where frequencies of cells with different guide RNAs are compared between different anatomical regions of the body; (3) screens based on single-RNA sequencing readout of the transcriptomic states of cells.

As such, in some cases, cells harvested from a tissue of interest are separated based on a phenotype of interest, such as expression of a protein of interest or a cellular activity measured by a fluorescent reporter or dye, and then guide RNAs are amplified in cell populations separated based on the property of interest, and compared to each other.

### Subject

The terms “individual” and “subject”, when referring to administering/introducing a subject nucleic acid (e.g., via virus such as AAV, lentivirus, and the like, or via non-virus such as nanoparticles, e.g., lipid nanoparticles) to a non-human animal, are used interchangeably herein, and refer to any convenient organism. In some cases, the subject is a non-human mammal, including, but not limited to, non-human primates, including simians; mammalian sport animals (e.g., horses); mammalian farm animals (e.g., sheep, goats, camel, alpaca, cows, pigs, etc.); mammalian pets (dogs, cats, etc.); and rodents (e.g., mice, rats, etc.). In some cases, the subject is a rodent (e.g., rat, mouse). In some cases, the subject is not mammalian (e.g., frog, fish, bird), and in some cases the subject is an invertebrate animal such as a fruit fly (e.g., Drosophila) or worm (C. elegans). In some cases, the subject is a plant. In some embodiments, the subject is an adult, in some cases a juvenile, and in some cases a newborn/neonate.

### Administration

Any convenient route of administration can be used to delivery/administer a subject nucleic acid (e.g., a guide RNA library) (e.g., an AAV, AAV library, etc.) to a subject, and one of ordinary skill in the art will be able to ready choose a route of delivery depending on the intended target tissue/organ and type of delivery used. A subject nucleic acid (e.g., via AAV, AAV library, etc.), can be administered systemically, regionally or locally, or by any route, for example, by injection, infusion, orally (e.g., ingestion or inhalation), or topically (e.g., transdermally). Possible delivery and administration methods can include parenteral, intravenous, intramuscular, intraperitoneal, intradermal, intracerebroventricular (ICV), subcutaneous, intracavity, intracranial, transdermal (topical), transmucosal and rectal administration. Example administration and delivery routes include intravenous, intraperitoneal, intraarterial, parenteral, subcutaneous, intra-pleural, topical, dermal, intradermal, transdermal, transmucosal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic. In some cases the delivery route is systemic (e.g., parenteral, intravenous). As such, in some embodiments, an AAV or AAV library is introduced into a subject via intravenous administration. In some cases, an AAV or AAV library is introduced into a subject via direct injection to an organ or tissue of the subject (e.g., intracerebroventricular (ICV) injection).

### Cell Marker

In some embodiments, a subject nucleic acid (e.g., an AAV nucleic acid) that comprises the probe cassette, i.e., the inverting probe plus the flanking recombinase target sites, and encodes the CRISPR/Cas guide RNA also includes a cell marker (e.g., a nucleotide sequence encoding fluorescent protein such as GFP (Green Fluorescent Protein), YFP (Yellow), RFP (Red), or BFP (Blue)). In some cases the cell marker is operably linked to a ubiquitous promoter such as an EF1a promoter. In some cases the fluorescent protein a nuclear localized BFP such as mTagBFP2. Such cell markers can allow visualization of cells that received the nucleic acid (e.g., AAV-transduced cells). Cell markers also include, but are not limited to selectable markers such as puromycin resistance, hygromycin resistance, blasticidine resistance, etc, which facilitate selecting cells to which guide RNAs were delivered by drug treatment.

Reagents, compositions, and kits/systems that find use in practicing the subject methods are also provided. For example, provided is a nucleic acid vector (e.g., an AAV nucleic acid vector) (and libraries of such vectors) that: (i) encodes a CRISPR/Cas guide RNA operably linked to a pol III promoter functional in a eukaryotic cell; (ii) encodes a cell marker operably linked to a promoter functional in the eukaryotic cell; and (iii) comprises an inversion cassette that comprises a probe sequence (e.g., spacer sequence) flanked by target sites for a recombinase protein. A library of such vectors is also provided, where the encoded CRISPR/Cas guide RNAs of the members of said library have different guide sequences from one another.

### Kits

Provided are kits/systems for carrying out a subject method. Such kits comprise various combinations of components useful in any of the methods described elsewhere herein. In some embodiments a subject kit includes: (1) a subject nucleic acid vector (e.g., AAV nucleic acid vector, e.g., one that includes a nucleic acid (flanked by ITRs) that: (a) encodes a CRISPR/Cas guide RNA operably linked to a promoter functional in a eukaryotic cell, and (b) includes an inversion cassette (a probe sequence flanked by target sites (e.g., Lox sites) for a recombinase (e.g., Cre));

and (2) a first and a second primer, where the first and second primers are specific for (hybridize to) sequences on the same DNA strand of the nucleic acid vector (e.g., AAV nucleic acid vector), where one of the primers hybridizes to the probe sequence (e.g., spacer sequence) such that upon inversion of the probe sequence (by the recombinase protein), the first and second primers form a primer pair. In some such cases, the other primer hybridizes to a sequence such that the guide RNA's guide sequence is positioned between the primer pair. In some cases, item (1) of the kit above is a library of such nucleic acid vectors (e.g., AAV nucleic acid vectors) —where the encoded CRISPR/Cas guide RNAs of the members of said library have different guide sequences from one another.

All of the above descriptions of the various components of the kit (e.g., the target sites, the recombinase, the ITRs, the probe sequence (e.g., spacer sequence), the CRISPR/Cas guide RNA, and the like) equally apply when the components are part of a kit. As an illustrative example, in some cases the recombinase is a Cre recombinase, in some cases, the target sites are Lox sites (in some cases mutated for unidirectional recombination), in some cases the CRISPR/Cas guide RNA is a CRISPR/Cas9 guide RNA, etc.

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method of verifying the introduction of a CRISPR/Cas guide RNA into a target cell of interest, the method comprising:
   (1) introducing into a target cell a nucleic acid that comprises a nucleotide sequence encoding a CRISPR/Cas guide RNA and comprises an inversion cassette comprising a probe sequence positioned between a first and a second target site for a site-specific recombinase,
   wherein, inside of the target cell, the CRISPR/Cas guide RNA is expressed and guides a CRISPR/Cas effector protein to a target sequence, and the recombinase (i) catalyzes inversion of the probe sequence, or (ii) catalyzes excision of the probe sequence to generate a neo-sequence;
   (2) extracting DNA from the target cell;
   (3) performing an amplification reaction with a primer pair, using the extracted DNA as a template, wherein one primer of the primer pair hybridizes to a sequence of the inverted probe sequence or to the neo-sequence, and the other primer hybridizes to a sequence such that the guide RNA's guide sequence is positioned between the primer pair, thereby resulting in amplification of the guide sequence,
   thereby verifying introduction of the CRISPR/Cas guide RNA into the target cell.
2. The method of 1, wherein said introducing comprises contacting the target cell with an AAV that infects said target cell, wherein the AAV comprises said nucleic acid.
3. The method of 1 or 2, wherein the nucleotide sequence encoding the CRISPR/Cas guide RNA is operably linked to a pol III promoter functional in the target cell.
4. The method of any one of 1-3, wherein the probe sequence is a spacer sequence.
5. The method of any one of 1-3, wherein the probe sequence comprises the sequence encoding the guide RNA.
6. The method of any one of 1-5, wherein the method is used for CRISPR-based in vivo screening, wherein:
(a) a library of said nucleic acids is introduced into a population of said target cells in a non-human subject; and
(b) the encoded CRISPR/Cas guide RNAs of the members of said library have different guide sequences from one another, such that the amplification reaction generates an sample of amplified guide sequences.
7. The method of 6, wherein prior to extracting DNA, a biological sample comprising the contacted population of target cells is collected from the subject in a range of from 1 day to 2 years after the library is introduced.
8. The method of 6 or 7, further comprising detecting and measuring quantities of guide sequences from the sample of amplified guide sequences to generate a test set of measured values.
9. The method of 8, wherein the test set of measured values is compared to a control set of measured values to determine whether any of the guide sequences are enriched or depleted in the test set relative to the control set.
10. The method of 8 or 9, wherein said detecting and measuring comprises high-throughput sequencing and quantification of the number of sequence reads for each detected guide sequence.
11. The method of any one of 7-10, wherein the biological sample comprises brain, heart, kidney, liver, lung, pancreas, retina, or skeletal muscle tissue.
12. The method of any one of 6-11, wherein the subject is an adult or a juvenile.
13. The method of any one of 6-11, wherein the subject is a neonate.
14. The method of any one of 6-11, wherein the subject is a rodent.
15. The method of any one of 6-14, wherein the subject is a mouse or a rat.
16. The method of any one of 1-15, wherein the recombinase is a Cre or Flp recombinase and the first and second target sites are Lox or FRT sites, respectively.
17. The method of 16, wherein the Lox or FRT sites are mutant sites for unidirectional recombination.
18. The method of any one of 1-15, wherein the recombinase is a phiC31 recombinase and the first and second target sites are attB and attP sites.
19. The method of any one of 1-18, wherein the CRISPR/Cas effector protein is Cas9.
20. The method of any one of 1-19, wherein the CRISPR/Cas effector protein is fused to a polypeptide functional domain that inhibits transcription (i.e., CRISPRi) or a polypeptide functional domain that activates transcription (i.e., CRISPRa).
21. The method of any one of 1-20, wherein the method further comprises introducing the recombinase and/or the CRISPR/Cas effector protein to the target cell, leading to said expression of the recombinase and/or the CRISPR/Cas effector protein.
22. The method of 21, wherein introducing the recombinase and/or the CRISPR/Cas effector protein comprises contacting the target cell with an AAV that delivers a nucleic acid encoding the recombinase and/or the CRISPR/Cas effector protein.
23. The method of any one of 1-22, wherein, prior to introducing said nucleic acid, the target cell comprises a nucleic acid encoding the recombinase and/or the CRISPR/Cas effector protein.
24. The method of any one of 1-23, wherein expression of the recombinase is controlled by a tissue specific promoter.
25. The method of any one of 1-23, wherein expression of the recombinase is controlled by a neuron-specific promoter.
26. The method of 25, wherein the neuron-specific promoter is an hSyn1 promoter.
27. The method of any one of 1-26, wherein the extracted DNA is episomal DNA.
28. The method of 2, wherein the serotype of the AAV's capsid is AAV-PHP.eB.
29. The method of any one of 1-28, wherein the probe sequence has a length of from 100-400 base pairs.
30. The method of any one of 6-29, wherein the introduction of said library comprises intravenous administration.
31. The method of any one of 6-29, wherein the introduction of said library comprises direct injection to an organ of the subject.
32. The method of any one of 6-29, wherein the introduction of said library comprises intracerebroventricular (ICV) injection.
33. The method of any one of 1-32, wherein the target cell is a non-dividing cell.
34. The method of any one of 1-32, wherein the target cell is a neuron.
35. The method of any one of 1-32, wherein the target cell is a brain cell.
36. A kit comprising:
(1) A nucleic acid comprising a nucleotide sequence encoding a CRISPR/Cas guide RNA and an inversion cassette that comprises a probe sequence positioned between a first and a second target site for a site-specific recombinase; and
(2) a first and a second primer, wherein the first and second primers hybridize to the same DNA strand of said nucleic acid, wherein one of the primers:
(i) hybridizes to the probe sequence such that upon inversion of the probe sequence by the recombinase, the first and second primers form a primer pair, or
(ii) hybridizes to a neo-sequence generated by excision of the probe sequence by the recombinase such that upon excision, the first and second primers form a primer pair; and
wherein the other primer hybridizes to a sequence such that the guide RNA's guide sequence is positioned between the primer pair.
37. The kit of 36, wherein the nucleic acid is an AAV nucleic acid vector.
38. The kit of 35 or 36, wherein the nucleotide sequence encoding the CRISPR/Cas guide RNA is operably linked to a pol III promoter.
39. The kit of any one of 36-38, wherein the probe sequence is a spacer sequence.
40. The kit of any one of 36-38, wherein the probe sequence comprises the sequence encoding the guide RNA.
41. The kit of any one of 36-40, wherein the nucleic acid further encodes a cell marker operably linked to a promoter.
42. The kit of any one of 36-41, wherein the recombinase is a Cre or Flp recombinase and the first and second target sites are Lox or FRT sites, respectively.
43. The kit of any one of 36-41, wherein the recombinase is a phiC31 recombinase and the first and second target sites are attB and attP sites.
44. The kit of any one of 36-43, wherein the CRISPR/Cas guide RNA is CRISPR/Cas9 guide RNA.
45. The kit of any one of 36-43, wherein the probe sequence has a length of from 100-400 base pairs.
46. A nucleic acid comprising:
(a) a nucleotide sequence encoding a CRISPR/Cas guide RNA,
(b) a nucleotide sequence encoding a cell marker, and
(c) an inversion cassette that comprises a probe sequence positioned between a first and a second target site for a site-specific recombinase.
47. The nucleic acid of 46, wherein the nucleic acid is an AAV nucleic acid vector.
48. The nucleic acid of 46 or 47, wherein the nucleotide sequence encoding the CRISPR/Cas guide RNA is operably linked to a pol Ill promoter.
49. The nucleic acid of any one of 46-48, wherein the probe sequence is a spacer sequence.
50. The nucleic acid of any one of 46-48, wherein the probe sequence comprises the sequence encoding the guide RNA.
51. The nucleic acid of any one of 46-50, wherein the recombinase is a Cre or Flp recombinase and the first and second target sites are Lox or FRT sites, respectively.
52. The nucleic acid of any one of 46-50, wherein the recombinase is a phiC31 recombinase and the first and second target sites are attB and attP sites
53. The nucleic acid of any one of 46-52, wherein the CRISPR/Cas guide RNA is CRISPR/Cas9 guide RNA.
54. The nucleic acid of any one of 46-53, wherein the probe sequence has a length of from 100-400 base pairs.
55. The nucleic acid of any one of 46-54, wherein the inversion cassette is positioned between the portion of the nucleic acid that encodes the CRISPR/Cas guide RNA and the portion of the nucleic acid that encodes the cell marker.
56. A library of the nucleic acids of any one of 46-55, wherein the encoded CRISPR/Cas guide RNAs of the members of said library have different guide sequences from one another.
57. A kit comprising:
   (1) a library of nucleic acids, wherein each nucleic acid comprises a nucleotide sequence encoding a CRISPR/Cas guide RNA and an inversion cassette that comprises a probe sequence positioned between a first and a second target site for a site-specific recombinase, and wherein the encoded CRISPR/Cas guide RNAs of the members of said library have different guide sequences from one another; and
   (2) a first and a second primer, wherein the first and second primers hybridize to the same DNA strand of said nucleic acid, wherein one of the primers:
(i) hybridizes to the probe sequence such that upon inversion of the probe sequence by the recombinase, the first and second primers form a primer pair, or
(ii) hybridizes to a neo-sequence generated by excision of the probe sequence by the recombinase such that upon excision, the first and second primers form a primer pair; and wherein the other primer hybridizes to a sequence such that the guide RNA's guide sequence is positioned between the primer pair.

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

### Results

Described here is a novel, scalable workflow for packaging and delivery of AAV that contains a pooled sgRNA library, and Cre-dependent isolation of episomal AAV DNA from tissues. This invention has broad applications for CRISPR-based screening, because Cre recombinase expression can be driven by promoters in specific tissues, cell-types, and at different times. Here, we provide a proof-of-concept for cell-type specific CRISPR-based screening—in this particular example the tissue is the mouse brain.

To allow for cell-type specific recovery of sgRNAs from homogenized brain tissue, we designed an AAV backbone to allow for Cre-lox driven recovery of PCR products in the presence of a cell type specific Cre, in this case, hSyn1 for neuronal expression. A diagram of the AAV plasmid backbone, designated as pAP215, is shown in FIG. 1. It contains an EF1a promoter-driven nuclear localized mTagBFP2, allowing visualization of transduced cells. It also expresses the sgRNA through an mU6 promoter. In this example embodiment, a region following the sgRNA sequence and scaffold contain a cassette flanked by lox71 and lox66 target sites. Specifically, the cassette is flanked by Lox71 and Lox66, which upon Cre recombination leads to an essentially irreversible switch to Lox72 and LoxP sequences. In short, using the pAP215 construct, Cre recombinase expression establishes a record of a cell having received the sgRNA and having expressed/contained the active Cre.

We next demonstrated that AAV episomes transduced in neurons could be recovered from whole brain tissue. We packaged the pAP215 backbone into AAV using the PHP.eB capsid, which allows robust transduction of neurons through intravenous delivery in mice. We intravenously delivered AAV of pAP215 with or without AAV to express Cre recombinase under a constitutive promoter (EF1a-Cre). Two weeks after delivery, we performed TRIzol-chloroform extraction from the whole brain, with RNA and AAV episomes predicted to be in the aqueous phase. Following RNase digest and column-based purification of the AAV episomes, we performed PCR to detect inversion of the Lox-flanked cassette using the primers as diagrammed in FIG. 1, which also shows the predicted product sizes. We detected evidence of the inverted plasmid only with expression of the Cre recombinase, confirming successful recovery of transduced AAV episomes from brain tissue (FIG. 1).

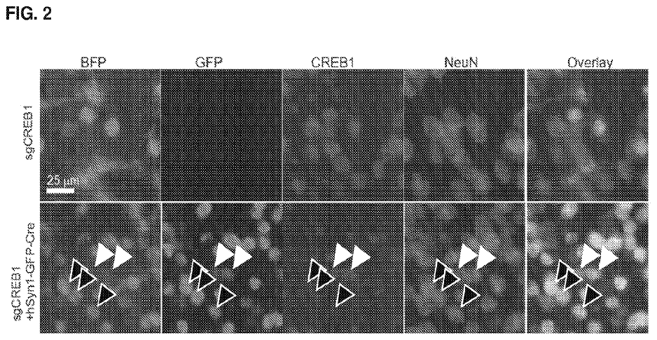

Next, we aimed to demonstrate whether the pAP215 can successfully knock down an endogenous gene by CRISPR interference (CRISPRi). CRISPR interference uses a catalytically inactive Cas9 protein that is fused to a strong transcriptional repressor; sgRNAs targeting the promoter of a gene recruits the CRISPRi machinery for gene suppression. We used a mouse harboring CRISPRi machinery conditionally expressed upon Cre recombinase expression, adding an additional level of control for cell-type selectivity and minimizing possible non-cell-autonomics effects. For a target gene, we selected Creb1, a nuclear transcription factor that is not essential for survival, and cloned an sgRNA targeting Creb1 into the pAP215 vector. For more robust and widespread CNS delivery of AAV, we performed intracerebroventricular (ICV) injection into neonates. After 3 weeks of expression, immunofluorescence staining of the brain tissue showed strong CREB1 knockdown in both sgRNA-BFP- and hSyn1-GFP-Cre-containing neurons only (FIG. 2). This supported that the pAP215 backbone can be used for CRISPRi-mediated knockdown of endogenous neuronal genes.

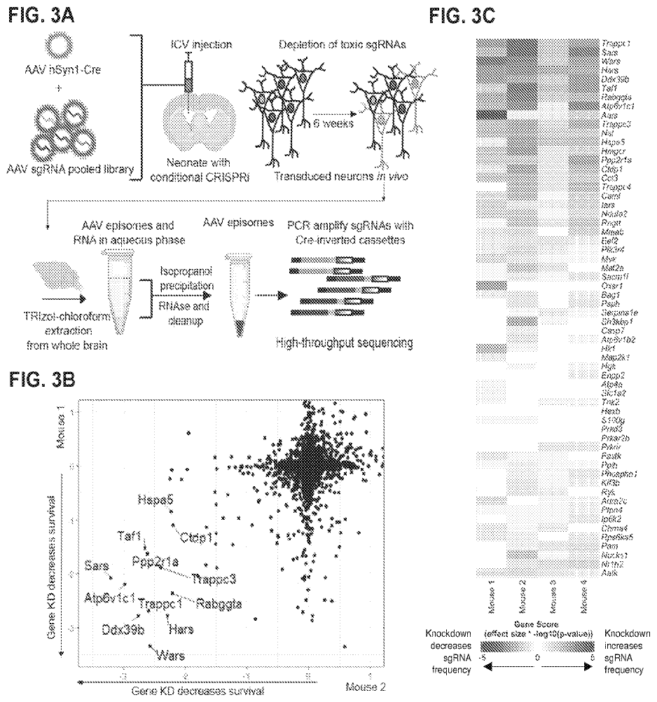

To establish the feasibility of a larger scale pooled CRISPRi screen, we cloned in a 12,350-element library targeting 2,269 genes and 250 non-targeting controls into pAP215. A workflow of the screen is diagrammed in FIG. 3A. This AAV pooled sgRNA library was injected by ICV in conditional CRISPRi neonates alongside AAV expressing Cre under a neuronal promoter (hSyn1). We expected that sgRNAs that knock down neuronal essential genes would be depleted relative to the starting population. Six weeks after the injection, AAV episomes were recovered, and sgRNAs were amplified using primers specific to the Lox-inverted cassette, and they were compared to the starting frequency of sgRNAs by sequencing the plasmid library pools.

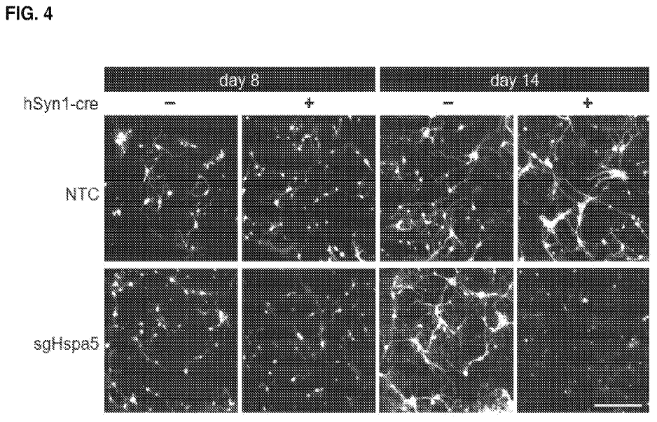

Analysis of the screen revealed several strong, consistent hits across multiple mice (FIG. 3B, 3C), with most hits decreasing neuronal survival. Several genes were clustered within certain families, such as tRNA synthetases (Wars, Hars, Sars) or endolysosomal pathway (Atp6v1c1, Rabggta). We tested the hit Hspa5 in primary neurons cultured from conditional CRISPRi mice for further validation. Expression of the sgRNA targeting Hspa5 led to marked Cre-dependent neuronal death within 2 weeks of expression (FIG. 4). This confirms the predictive capability of the in vivo AAV- and CRISPR-based screening approach to define genes with tissue specific effects. In this particular case essential genes were identified in neurons, but this example shows that any desired cell-type (e.g., any desired tissue or organ) can be targeted, and any desired phenotype can be assessed.

### CITATIONS

1. Chow R D, Guzman C D, Wang G, et al. AAV-mediated direct in vivo CRISPR screen identifies functional suppressors in glioblastoma. Nat Neurosci. 2017; 20(10):1329-1341. doi: 10.1038/nn.4620
2. Wertz M H, Mitchem M R, Pineda S S, et al. Genome-wide In Vivo CNS Screening Identifies Genes that Modify CNS Neuronal Survival and mHTT Toxicity. Neuron. 2020; 106(1):76-89.e8. doi: 10.1016/j.neuron.2020.01.004
3. Ruetz T J, Kashiwagi C M, Morton B, et al. In vitro and in vivo CRISPR-Cas9 screens reveal drivers of aging in neural stem cells of the brain. Published online Nov. 23, 2021:2021.11.23.469762. doi:10.1101/2021.11.23.469762
4. Chen X, Ravindra Kumar S, Adams C D, et al. Engineered AAVs for non-invasive gene delivery to rodent and non-human primate nervous systems. Neuron. 2022; 110(14):2242-2257.e6. doi: 10.1016/j.neuron.2022.05.003
5. Wang G, Chow R D, Ye L, et al. Mapping a functional cancer genome atlas of tumor suppressors in mouse liver using AAV-CRISPR-mediated direct in vivo screening. Sci Adv. 2018; 4(2):eaao5508. doi:10.1126/sciadv.aao5508
6. Horlbeck M A, Gilbert L A, Villalta J E, et al. Compact and highly active next-generation libraries for CRISPR-mediated gene repression and activation. eLife. 2016; 5:e19760. doi:10.7554/eLife.19760
7. Negrini M, Wang G, Heuer A, Björklund T, Davidsson M. AAV Production Everywhere: A Simple, Fast, and Reliable Protocol for In-house AAV Vector Production Based on Chloroform Extraction. Curr Protoc Neurosci. 2020; 93(1):e103. doi: 10.1002/cpns.103
8. Kim J Y, Grunke S D, Levites Y, Golde T E, Jankowsky J L. Intracerebroventricular Viral Injection of the Neonatal Mouse Brain for Persistent and Widespread Neuronal Transduction. JoVE J Vis Exp. 2014; (91):e51863. doi:10.3791/51863
9. Tian R, Gachechiladze M A, Ludwig C H, et al. CRISPR Interference-Based Platform for Multimodal Genetic Screens in Human iPSC-Derived Neurons. Neuron. 2019; 104(2):239-255.e12. doi:10.1016/j.neuron.2019.07.014

### Sequences Used in Example 1

| | | SEQ ID |
|---|---|---|
| Description | Sequence | NO: |
| Spacer sequence | ACATGTAAACGATCCTAGGCGTCCATAT | 1 |
| (Sequence between LoX sites) | GATGCATGGCAGCAATTGGCCATTTGTA | |
| | GGCAGACGCATCGATCAGTACAGTCGG | |
| | ATAGTCGAACGATCATTACAGTCAGTCG | |
| | AATGCAAAGTAGCGAGGCATGACCTGT | |
| | GTACTTAAAGCATGACTGGTACTGACAC | |
| | GTCGCATGC | |
| Forward Primer | caagcagaagacggcatacgaGATgcacaaaagga | 2 |
| for sequencing | aactcaccct | |
| (oMK723_HS4Kmirror_ | | |
| CRISPR_rev) | | |
| Portion of primer | TGACTGGTACTGACACGTCG | 3 |
| Binding to the | | |
| recombination site | | |
| Mirror Primers | | |
| (reverse) with | | |
| Illumina Index | | |
| Mouse_i1 | aatgatacggcgaccaccgaGATCTACACGATC | 4 |
| | GGAAGAGCACACGTCTGAACTCCAGTC | |
| | ACATCACGTGACTGGTACTGACACGTCG | |
| Mouse_i2 | aatgatacggcgaccaccgaGATCTACACGATC | 5 |
| | GGAAGAGCACACGTCTGAACTCCAGTC | |
| | ACCGATGTTGACTGGTACTGACACGTCG | |
| Mouse_i3 | aatgatacggcgaccaccgaGATCTACACGATC | 6 |
| | GGAAGAGCACACGTCTGAACTCCAGTC | |
| | ACTTAGGCTGACTGGTACTGACACGTCG | |
| Mouse_i4 | aatgatacggcgaccaccgaGATCTACACGATC | 7 |
| | GGAAGAGCACACGTCTGAACTCCAGTC | |
| | ACTGACCATGACTGGTACTGACACGTCG | |
| Mouse_i5 | aatgatacggcgaccaccgaGATCTACACGATC | 8 |
| | GGAAGAGCACACGTCTGAACTCCAGTC | |
| | ACACAGTGTGACTGGTACTGACACGTCG | |
| Mouse_i6 | aatgatacggcgaccaccgaGATCTACACGATC | 9 |
| | GGAAGAGCACACGTCTGAACTCCAGTC | |
| | ACGCCAATTGACTGGTACTGACACGTCG | |
| Mouse_i7 | aatgatacggcgaccaccgaGATCTACACGATC | 10 |
| | GGAAGAGCACACGTCTGAACTCCAGTC | |
| | ACCAGATCTGACTGGTACTGACACGTCG | |
| mouse_i19 | aatgatacggcgaccaccgaGATCTACACGATC | 11 |
| | GGAAGAGCACACGTCTGAACTCCAGTC | |
| | ACGTGAAATGACTGGTACTGACACGTCG | |
| mouse_20 | aatgatacggcgaccaccgaGATCTACACGATC | 12 |
| | GGAAGAGCACACGTCTGAACTCCAGTC | |
| | ACGTGGCCTGACTGGTACTGACACGTC | |
| | G | |
| mouse_i21 | aatgatacggcgaccaccgaGATCTACACGATC | 13 |
| | GGAAGAGCACACGTCTGAACTCCAGTC | |
| | ACGTTTCGTGACTGGTACTGACACGTCG | |
| mouse_i23 | aatgatacggcgaccaccgaGATCTACACGATC | 14 |
| | GGAAGAGCACACGTCTGAACTCCAGTC | |
| | ACGAGTGGTGACTGGTACTGACACGTC | |
| | G | |
| mouse_i8 | aatgatacggcgaccaccgaGATCTACACGATC | 15 |
| | GGAAGAGCACACGTCTGAACTCCAGTC | |
| | ACACTTGATGACTGGTACTGACACGTCG | |
| mouse_i10 | aatgatacggcgaccaccgaGATCTACACGATC | 16 |
| | GGAAGAGCACACGTCTGAACTCCAGTC | |
| | ACTAGCTTTGACTGGTACTGACACGTCG | |
| mouse_i11 | aatgatacggcgaccaccgaGATCTACACGATC | 17 |
| | GGAAGAGCACACGTCTGAACTCCAGTC | |
| | ACGGCTACTGACTGGTACTGACACGTC | |
| | G | |
| mouse i12 | aatgatacggcgaccaccgaGATCTACACGATC | 18 |
| | GGAAGAGCACACGTCTGAACTCCAGTC | |
| | ACCTTGTATGACTGGTACTGACACGTCG | |
| mouse_i13 | aatgatacggcgaccaccgaGATCTACACGATC | 19 |
| | GGAAGAGCACACGTCTGAACTCCAGTC | |
| | ACAGTCAATGACTGGTACTGACACGTCG | |
| mouse_i14 | aatgatacggcgaccaccgaGATCTACACGATC | 20 |
| | GGAAGAGCACACGTCTGAACTCCAGTC | |
| | ACAGTTCCTGACTGGTACTGACACGTCG | |
| mouse_i25 | aatgatacggcgaccaccgaGATCTACACGATC | 21 |
| | GGAAGAGCACACGTCTGAACTCCAGTC | |
| | ACACTGATTGACTGGTACTGACACGTCG | |
| mouse_i27 | aatgatacggcgaccaccgaGATCTACACGATC | 22 |
| | GGAAGAGCACACGTCTGAACTCCAGTC | |
| | ACATTCCTTGACTGGTACTGACACGTCG | |

### Methods

### Design of Screening Construct

The screening vector pAP215 diagrammed in FIG. 1 was generated using the pAAV-U6-sgRNA-CMV-GFP plasmid (Addgene #85451) as the starting backbone. We replaced the hU6-sgRNA cassette with the mU6-sgRNA cassette from pMK1334 (Addgene #127965) by restriction cloning between sites MluI and XbaI. The mU6 was then replaced using Gibson Assembly with a gene block (gBlock, IDT technologies) containing a modified mU6 which combined the first 303

(gatccgacgccgccatctctaggcccgcgccggccccctcgcac
agacttgtgggagaagctcggctactcccctgccccggttaattt
gcatataatatttcctagtaactatagaggcttaatgtgcgataa
aagacagataatctgttctttttaatactagctacattttacatg
ataggcttggatttctataagagatacaaatactaaattattatt
ttaaaaaacagcacaaaaggaaactcaccctaactgtaaagtaat
tgtgtgttttgagactataaatatcccttggaga
(SEQ ID NO: 23))

nucleotides from a separate mU6 sequence derived from pMU6-gRNA and the latter 11 nucleotides contained a BstXI restriction site (ccaccttgttg (SEQ ID NO: 24)). The CMV-EGFP was replaced by EF1a-2xmycNLS-tagBFP2 from pMK1334 by Gibson Assembly. The W3 terminator was inserted at the EcoRI-Xhol multiple cloning site by Gibson assembly and the sequence was cloned from Cbh_v5 AAV-saCBE C-Terminal plasmid (Addgene #137138). The hGH was replaced by the SV40 from pMK1334. The Lox66 and Lox71 sequences and orientation were copied from the pFrt-invCAG-Luc (Addgene #63577) and were cloned in along with the 175-bp inverting spacer between the LoxP sites by gBlock at the XbaI-KpnI multiple cloning site.
sgRNA Cloning

To generate a functional mouse sgRNA pooled library of the pAP215 plasmid, we transferred the sgRNA sequences from the published CRISPRi V2 M1 plasmid library6 (Addgene #83998), kindly provided by the Gilbert Lab at UCSF. 20 μg of the library was digested with BstXI (Thermo, cat. no. ER1021) and Bpu1102I (Thermo cat. no. ER0091). The insert guides (84 bp) were resolved on a 4-20% TBE gel (Thermo cat. no. EC62252BOX) and precipitated with GlycoBlue and sodium acetate. Insert guides were washed with ethanol after precipitation and then eluted in DNase- and RNase-free autoclaved MilliQ water. The backbone vector, pAP215, was digested in parallel with BstXI and BlpI, resolved on a 1% agarose gel, and purified from the gel (Zymo Research cat. no. D4001). The vectors and insert guides were annealed for 16 hr overnight use T4 ligase at a 1:2 ratio of vector to insert, and then purified with sodium acetate and ethanol washing. After the final wash, the library product was transformed into Takeda commercial chemically competent E. coli, and 10 colonies were picked at random to ensure that each colony was unique. Upon confirmation, the library product was electroporated into Mega-X competent cells (Thermo cat. no. C640003) and grown overnight, and a portion of the culture was plated to determine if at least 250 colonies per guide was achieved, followed by growth of the remainder of the in 1 L of LB, and purification of the library using ZymoPURE II Plasmid Gigaprep Kit (Zymo Research cat. no. D4204).

Individual sgRNAs were cloned in the pAP215 backbone digested with BstXI and BlpI using annealed oligos. The protospacer sequences for the specific sgRNAs used in this study include: sgCreb1 (5′ GGCTGCGGCTCCTCAGTCGG 3′ (SEQ ID NO: 25)), sgHspa5 (5′ GAACACTGACCTGGACACTT 3′ (SEQ ID NO: 26)), and a non-targeting control (5′ GGATGCATAGATGAACGGATG 3′ (SEQ ID NO: 27))

### AAV Packaging and Purification

The pAP215-M1 library was packaged into AAV for transduction into neonatal mice as follows. Two 15-cm dishes were each seeded with 1.5×10⁷ HEK 293T cells (ATCC CRL-3216) in 25 mL DMEM supplemented with 10% FBS, 1% penicillin/streptomycin, and GlutaMAX™ (Thermo cat. no. 35050061). The next day, 20 μg of pAdDeltaF6 (Addgene #112867), 7 μg of M1 library plasmid, 7 μg of pUCmini-iCAP-PHP.eB (Addgene #103005), and 75 μL of 1 mg/ml polyethenylamine (PEI) (Linear, MW 25,000, Polysciences 23966) were diluted into 4 ml of OptiMEM™ (Thermo 31985062), gentle mixed, and incubated at room temp for 10 min. The PEI/DNA transfection complex was then pipetted drop-wise onto the HEK 293T cells. After 24 hours the media was replaced with fresh 27 mL Opti-MEM™.

AAV precipitation was performed as previously described⁷, with modifications. Cold 5x AAV precipitation solution (40% polyethylene glycol (Sigma cat. 89510) and 2.5 M NaCl) was previously prepared. The cells and media were triturated and collected (~30 ml) into a 50 ml conical tube, followed by addition of 3 ml chloroform and vortexing for approximately 30 seconds. The homogenate was centrifuged at 3000 g×5 min at RT, and the aqueous (top) phase was transferred to a new 50 ml conical tube and with AAV precipitation solution diluted to 1×, followed by incubation on ice for at least 1 hour. The solution was centrifuged at 3000 g×30 min at 4° C. The supernatant was completely removed and the viral pellet was resuspended in 1 ml of 50 mM HEPES and 3 mM MgCl₂, and incubated with 1 μl DNase I (New England Biolabs, cat. M0303S) and 10 μl RNase A (Thermo Fisher Scientific, cat. no. EN0531) at 37° C. for 15 min. A 1:1 volume of chloroform was added, followed by vortexing for 15 sec, and centrifuged at 16,000 g×5 min at RT. Using 400 ml at a time, the aqueous phase was completely flowed through a 0.5-ml Amicon Ultra Centrifugal Filter, 100 kDa (Millipore, cat. no. UFC510024) with 14,000 g×3 min centrifugation, followed by washing twice with 1×DPBS.

To prepare AAV for testing in primary neuronal cultures, HEK293T cells were seeded into a 6-well format containing 1.5 ml of media. The cells were transfected with 1 mg helper, 350 ng PHP.eB, and 350 ng of AAV transfer plasmid as above. Approximately 48 hours after transfection, the cells and media were collected in 2 ml microfuge tube, 200 ml of chloroform was added to each tube, vortexed for 15 sec, and centrifuged at 16,000 g×5 min RT. The aqueous (top) phase was transferred to a new tube and AAV precipitation solution was added to 1x dilution, and incubated on ice for at least one hour. The precipitated AAV was centrifuged at 16,000 g×15 min at 4° C., the supernatant was removed, the pellet was resuspended in 100 ml of 1×PBS, and centrifuged again at 16,000 g×1 min to remove excess debris, and the supernatant (purified virus) was transferred to a new microfuge tube. 10 μl purified virus was used per well in primary neuronal cultures in a 24-well format.

### Animals

All animal procedures were conducted in accordance with UCSF Institutional Animal Care and Use Committee. Mice were housed on a 12-h light/dark cycle at 22-25° C., 50-60% humidity, and had food and water provided ad libitum. LSL-dCas9-KRAB mice (B6;129S6-Gt(ROSA)26Sor^{tm2(CAG-cas9*/ZNF10*)Gers}/J, RRID: IMSR_JAX:033066.) were kindly provided by the laboratory of Dr. Suneil Koliwad at UCSF.

### Retroorbital Injection

10 μl of AAV was diluted into 100 μl of 1×DPBS and loaded into a 1 ml 30-gauge insulin syringe with 0.5 inch needle length. Mice were anesthetized in a chamber containing 0.5 ml isoflurane for approximately 10 seconds. The full volume was injected into the right retroorbital space for each mouse, and the mice were returned to their cage after awakening.

### Intracerebroventricular Injection

Intracerebroventricular (ICV) injections were performed as previously described with minor modifications⁸. Briefly, neonates were placed on a gauze-covered frozen cold pack and monitored for complete cryoanesthesia. The scalp was gently cleaned with an alcohol swab. 2 μl of AAV with 0.1% trypan blue was loaded into Hamilton Model 1701 10 μl syringe (Fisher Scientific Cat #14-824-652). The syringe was equipped with a 33-gauge beveled needle (Hamilton, 0.5 inches in length). The needle was inserted through the skull ⅖ of distance of the lambda suture to the eye and to a depth of 3 mm to target the left lateral ventricle. Following a one-time unilateral injection, the neonate was placed on a warming pad for recovery and returned to the parent cage.

sgRNA Recovery, Sequencing, and Analysis

Animals were euthanized using CO₂, and their whole brains were removed and stored at −80° C. Each brain was placed in a Dounce Homogenizer (Thomas Scientific 1234F37 Mfr. No. 7722-7) with 2 ml of TRIzol™ (Thermo cat. 15596026) and thoroughly homogenized. The homogenate was divided into two 1.5 ml centrifuge tubes and 0.2 ml of chloroform was added to each tube, followed by centrifugation at 12000 g for 15 min at 4° C. The aqueous phase was transferred to a new tube, and 0.5 ml of isopropanol was added and centrifuged at 12000 g for 10 min. The supernatant was discarded and the pellet was resuspended in 1 mL of 75% ethanol in DNase/RNase-free H₂O, then vortexed briefly, and then spun down at 7500 g×5 min. The supernatant was then removed and the pellet was allowed to air dry for 10 minutes, and then resuspended in 100 μl of DNase/RNase-free H₂O and incubated with 1 μl of RNase A (Thermo EN0531) at 37° C. overnight. The sample was then column purified (Zymo DNA Clean & Concentrator) and eluted in 50 μl of DNase/RNase-free H₂O to yield recovered viral DNA.

The purified episomal DNA and the starting plasmid pooled library were PCR amplified with adapter sequences, sequenced on the Illumina HiSeq4000 at the UCSF Center for Advanced Technology, and analyzed as previously described⁹. Phenotypes and p-values for each gene were generated using the following pipeline described on our laboratory website (https://kampmannlab.ucsf.edu/mageck-inc).

### Mouse Cortical Neuron Primary Cultures

Neonates were briefly sanitized with 70% EtOH and decapitated using sharp scissors, and the brains were removed and placed into cold HBSS (Thermo cat. no. 14175095). The meninges were removed under a dissecting microscope, and the cortices were transferred to a 15-ml conical tube containing 10 ml of 0.25% Trypsin-EDTA (Thermo cat. no. 25200056) and incubated at 37° C. for 30 min. The trypsin was removed and the brains were gently rinsed twice in 5 ml of DMEM complete media, followed by trituration of brains in 5 ml of DMEM complete media using a glass Pasteur pipette (VWR cat. no. 14672-380). The triturated tissue was resuspended of DMEM complete media and filtered through a 40 mm cell strainer (Corning™ cat. no. 352340). Approximately one brain was plated across each Corning® BioCoat® Poly-D-Lysine 24-well Clear Flat Bottom TC-treated Multiwell Plates (cat. no. 356414). The following day (DIV1), the DMEM complete media was replaced with neuronal growth media composed of Neurobasal™-A Medium (Thermo cat. no. 10888022), B-27™ Supplement without vitamin A (Thermo cat. 12587010), GlutaMAX™, and 1% penicillin/streptomycin. On DIV2, the cultures were further supplemented with cytarabine to a final concentration of 1 mM (Thermo cat. no. 449561000).

The primary neuronal cultures were transduced with AAV on DIV4 and imaged starting on 4 days after transduction, every 2 days, using the ImageXpress Micro Confocal High-Content Imaging System (Molecular Devices).

### Immunofluorescence Staining of Mouse Brain

Whole brains were fixed overnight in 4% paraformaldehyde (Electron Microscopy Sciences, 15710) diluted in 1×PBS. The following day, the fixative was replaced with 30% sucrose dissolved in 1×PBS for cryoprotection for at least 2 days. The brains were sectioned in the sagittal plane at 40 μm and the slices were stored in 1×PBS+0.05% NaN₃ at 4° C. Representative brain slices were incubated in blocking solutions (1×PBS containing 10% normal goat serum, 0.3% Triton X-100, and 1% BSA) for one hour at room temperature. The slices were incubated in primary antibody overnight with the following: 1:1000 rabbit anti-CREB 48H2 (Cell Signaling, 9197), 1:500 guinea pig anti-NeuN Synaptic Systems, 266004). The tissues were gently washed 3×10 min in 1×PBS, incubated in secondary antibody. The cells were briefly washed three times with 1×PBS and incubated with 1:1000 secondary antibody (AlexaFluor, Thermo) diluted in blocking solution for 2 hours at room temperature.

### Example 2

### Results

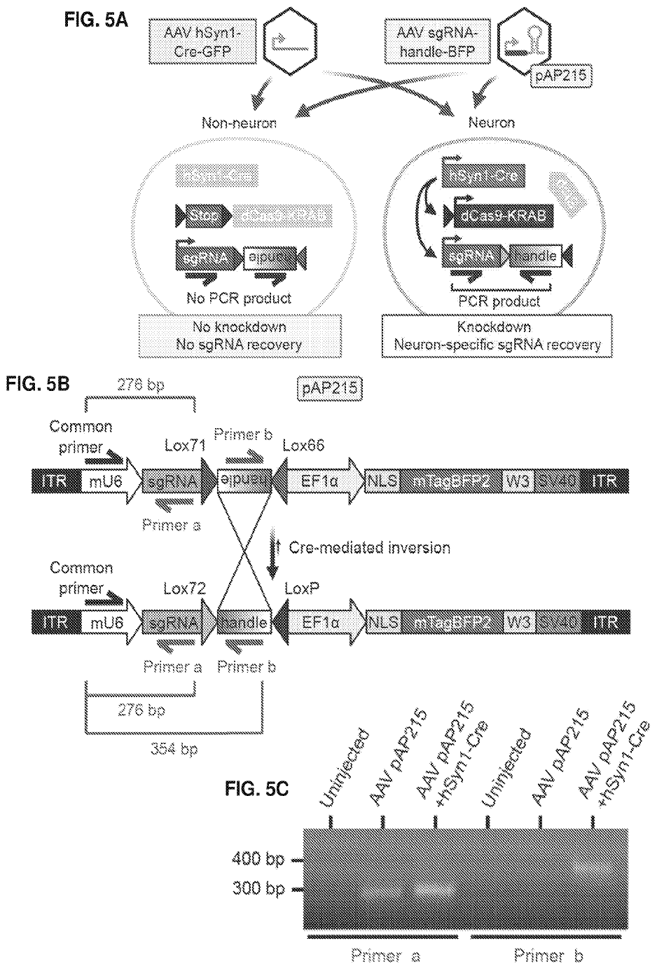

FIG. 5A-5C. AAV backbone for cell type-specific, cre-dependent sgRNA recovery. FIG. 5A, Strategy for cell-type selective screening. FIG. 5B, The pAP215 AAV sgRNA backbone contains a Lox71/Lox66 flanked “handle” that undergoes inversion only in cells expressing Cre recombinase, allowing for cell type-specific sgRNA recovery by PCR. FIG. 5C, PCR performed on episomes recovered from mouse brains expressing AAV pAP215 with or without hSyn1-Cre-NLS-GFP (Cre) using the primers diagrammed in (B). PCR amplicons from brains of mice injected by ICV at P0 with pPA215 packaged into AAV PHP.eB, with or without co-injection of hSyn1-Cre packaged into AAV PHP.eB. The first three lanes represent samples from uninjected brain, pAP215 alone, and pAP215+hSyn1-Cre shows amplicons generated with Primer A, which captures presence of AAV episomes, but is not inversion specific. The next three lanes represent amplicons from the same samples generated with Primer B, which binds to the lox-flanked recombination site (handle) and will generate amplicons only after cre-mediated handle inversion.

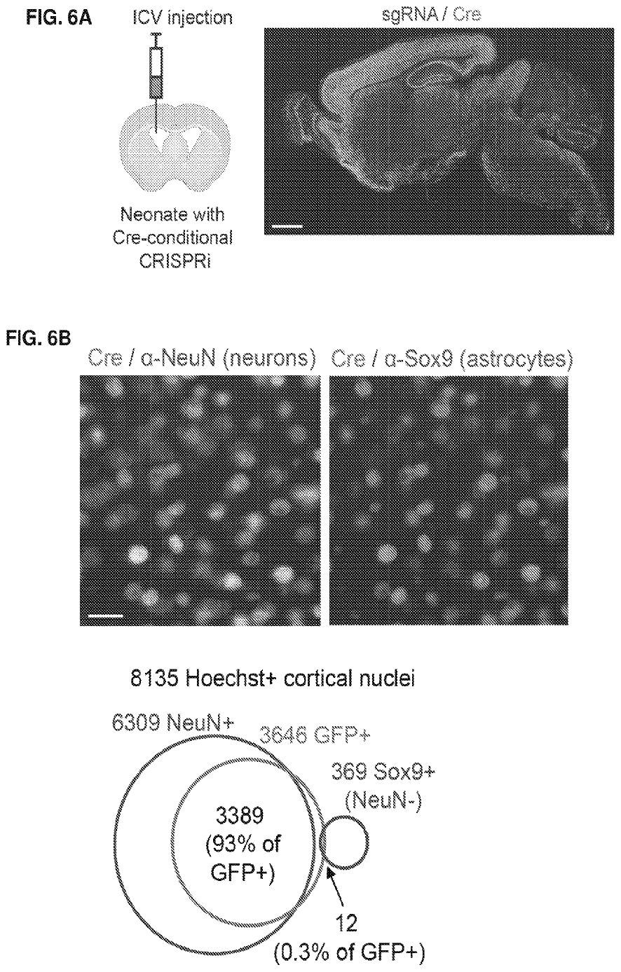

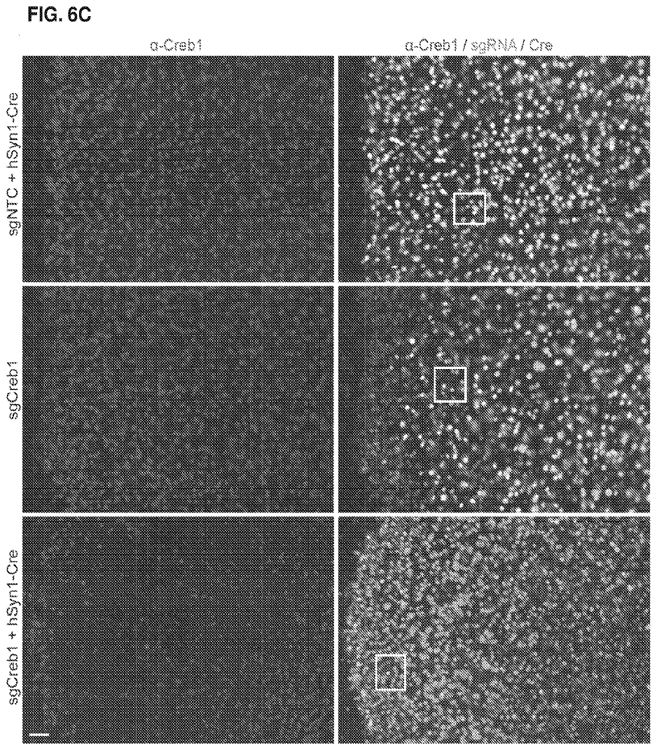

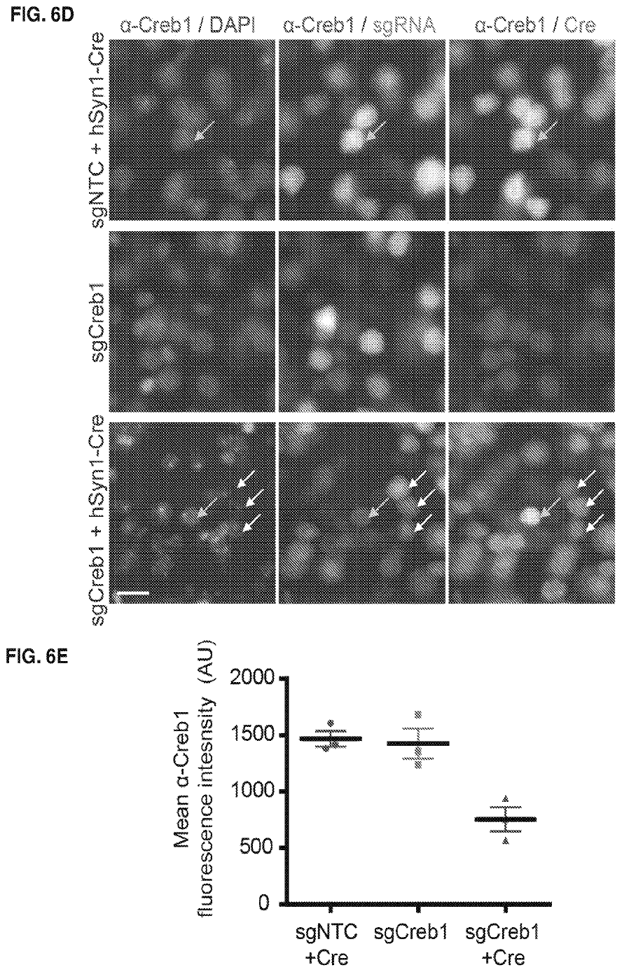

FIG. 6A-6E. Cre-dependent CRISPRi knockdown of endogenous genes in neurons throughout the mouse brain. FIG. 6A, Expression of AAV-pAP215 (sgRNA, based on BFP marker) and AAV-hSyn1-Cre-NLS-GFP (Cre, based on GFP) in a mouse brain three weeks after neonatal intracerebroventricular (ICV) delivery. Scale bar, 1 mm. FIG. 6B, Immunofluorescent staining for neurons (marker: NeuN) and astrocytes (marker: Sox9) in mouse cortex expressing AAV-hSyn1-Cre-NLS-GFP. Scale bar, 25 μm. Overlap of Cre expression with NeuN and Sox9 within a cortical region demonstrate high specificity for neurons and high coverage of neurons. FIG. 6C, sgRNAs targeting Creb1 (sgCreb1) or a non-targeting control (sgNTC) in AAV-pAP215 were delivered with or without AAV hSyn1-Cre-NLS-GFP via ICV injection into neonatal mice with lox-stop-lox CRISPRi machinery. Brains were stained at 3 weeks with a representative area of frontal cortex shown. Scale bar, 50 μm. FIG. 6D, Higher magnification of the boxed regions in the left panels, with white arrows indicating examples of neurons having received both sgCreb1 and Cre, whereas yellow arrows indicate neurons that received Cre without sgCreb1. Scale bar, 10 μm. FIG. 6E, Quantification of Creb1 levels in sgRNA-positive nuclei within a representative region of cortex (mean±s.e.m, n=3 independent mice).

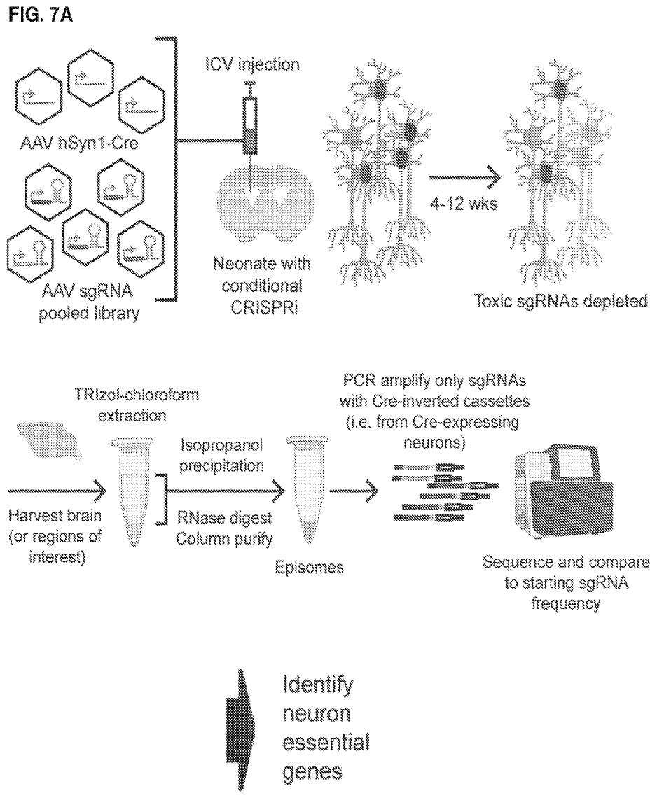

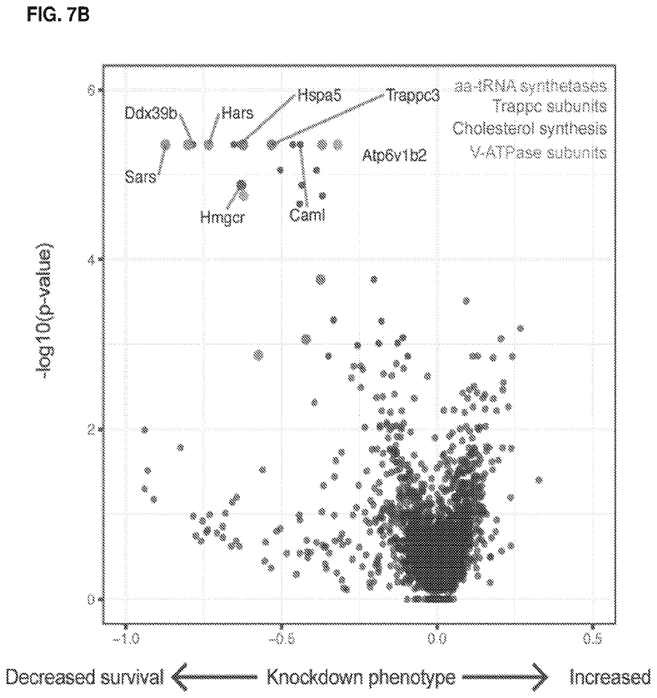

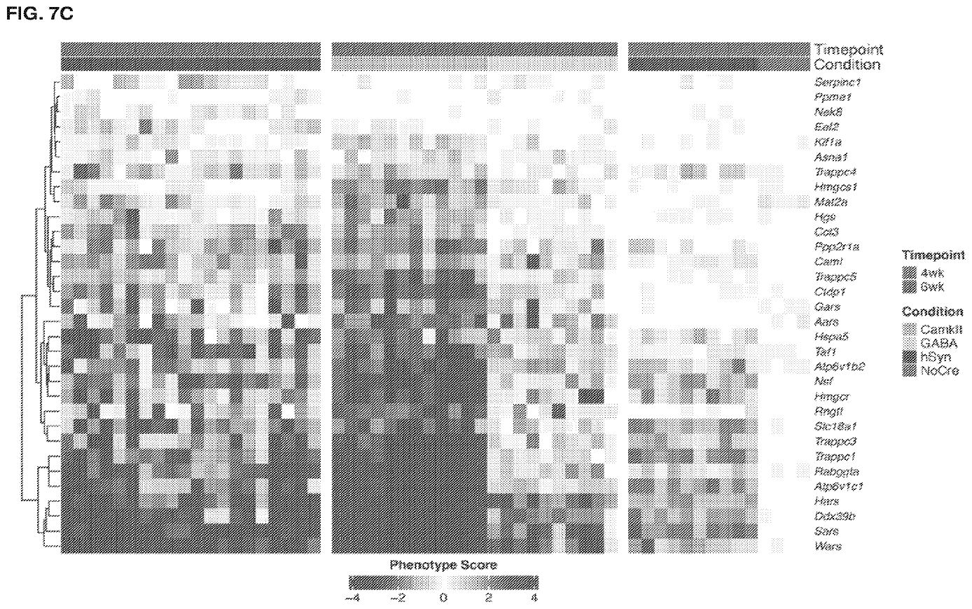

FIG. 7A-7C. in vivo CRISPRi screen for neuron-essential genes in mouse brains. FIG. 7A, Strategy for in vivo CRISPRi screening to identify neuron-essential genes. FIG. 7B, Knockdown phenotypes for 2,269 genes averaged across n=20 mice at 6 weeks of age after ICV injection of an sgRNA library. Genes with an FDR <0.1 are colored blue, and genes within enriched pathways are further color-coded. FIG. 7C, Phenotypes of neuron-essential genes (rows) from (B) with an FDR <0.1 in individual mice (columns), including with Cre recombinases driven under different promoters, and with and without Cre recombinase expression.

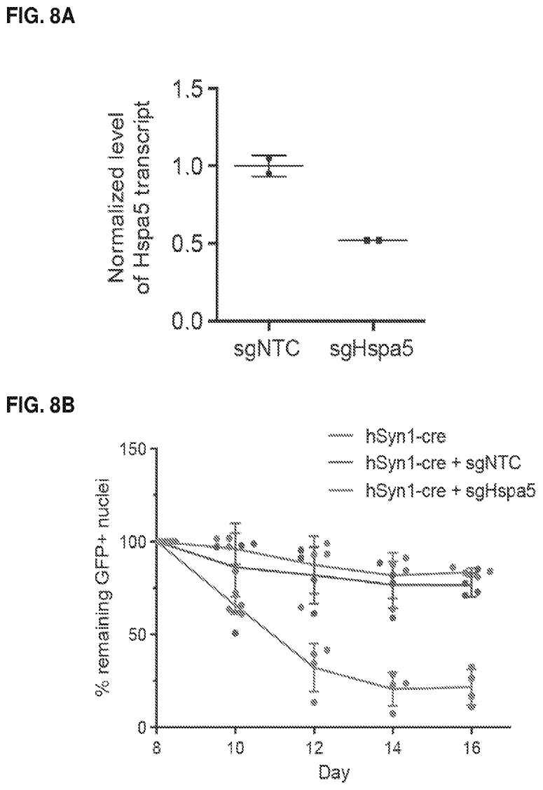

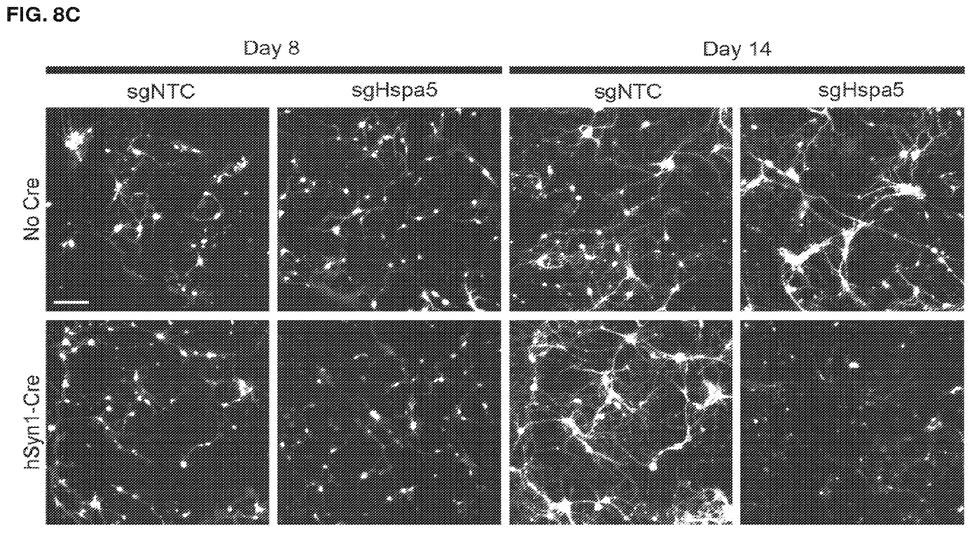

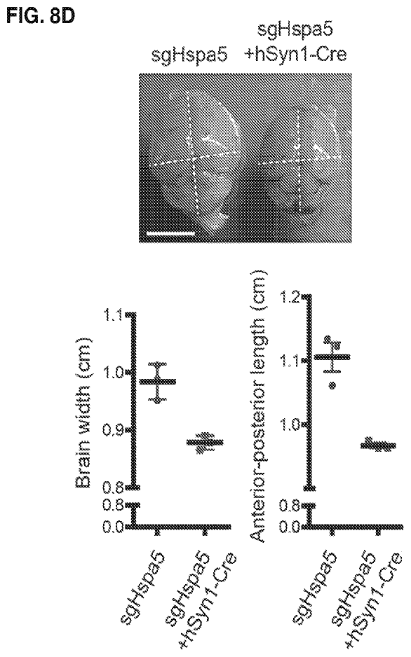

FIG. 8A-8D. Validation of Hspa5 as an essential gene in mouse neurons. FIG. 8A, Cultured mouse embryonic fibroblasts (MEFs) from constitutive dCas9-KRAB mice were transduced with lentivirus (pMK1334) containing an sgRNA targeting Hspa5 (sgHspa5) versus a non-targeting control sgRNA (sgNTC). Hspa5 mRNA levels were assayed by qPCR and normalized to the sgNTC control (mean±s.d., n=2 technical replicates). FIG. 8B, Survival of primary neurons cultured from conditional CRISPRi mice following transduction with AAV-hSyn1-Cre-NLS-GFP alongside AAV-AP215 containing sgHspa5 versus sgNTC (mean±s.d., n=4 wells). FIG. 8C, Representative images of primary neurons at 8 or 14 days post transduction with the AAVs as in panel B. Cells were co-transduced with constitutively expressed cytosolic mScarlet, which reveals fine neuronal processes, displayed in this panel. Scale bar 125 μm. FIG. 8D, Representative brains of conditional CRISPRi mice 16 days after neonatal ICV injection of AAV-pAP215-sgHspa5 with or without hSyn1-Cre-NLS-GFP, and brain measurements (mean±s.d., n=3 independent mice). Scale bar, 5 mm.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of verifying the introduction of a CRISPR/Cas guide RNA into a target cell of interest, the method comprising:
(1) introducing into a target cell a nucleic acid that comprises a nucleotide sequence encoding a CRISPR/Cas guide RNA and comprises an inversion cassette comprising a probe sequence positioned between a first and a second target site for a site-specific recombinase,
wherein, inside of the target cell, the CRISPR/Cas guide RNA is expressed and guides a CRISPR/Cas effector protein to a target sequence, and the recombinase (i) catalyzes inversion of the probe sequence, or (ii) catalyzes excision of the probe sequence to generate a neo-sequence;
(2) extracting DNA from the target cell;
(3) performing an amplification reaction with a primer pair, using the extracted DNA as a template, wherein one primer of the primer pair hybridizes to a sequence of the inverted probe sequence or to the neo-sequence, and the other primer hybridizes to a sequence such that the guide RNA's guide sequence is positioned between the primer pair, thereby resulting in amplification of the guide sequence,
thereby verifying introduction of the CRISPR/Cas guide RNA into the target cell.

2. The method of claim 1, wherein said introducing comprises contacting the target cell with an AAV that infects said target cell, wherein the AAV comprises said nucleic acid.

3. The method of claim 1 or claim 2, wherein the nucleotide sequence encoding the CRISPR/Cas guide RNA is operably linked to a pol Ill promoter functional in the target cell.

4. The method of any one of claims 1-3, wherein the probe sequence is a spacer sequence.

5. The method of any one of claims 1-3, wherein the probe sequence comprises the sequence encoding the guide RNA.

6. The method of any one of claims 1-5, wherein the method is used for CRISPR-based in vivo screening, wherein:
(c) a library of said nucleic acids is introduced into a population of said target cells in a non-human subject; and
(d) the encoded CRISPR/Cas guide RNAs of the members of said library have different guide sequences from one another, such that the amplification reaction generates an sample of amplified guide sequences.

7. The method of claim 6, wherein prior to extracting DNA, a biological sample comprising the contacted population of target cells is collected from the subject in a range of from 1 day to 2 years after the library is introduced.

8. The method of claim 6 or claim 7, further comprising detecting and measuring quantities of guide sequences from the sample of amplified guide sequences to generate a test set of measured values.

9. The method of claim 8, wherein the test set of measured values is compared to a control set of measured values to determine whether any of the guide sequences are enriched or depleted in the test set relative to the control set.

10. The method of claim 8 or claim 9, wherein said detecting and measuring comprises high-throughput sequencing and quantification of the number of sequence reads for each detected guide sequence.

11. The method of any one of claims 7-10, wherein the biological sample comprises brain, heart, kidney, liver, lung, pancreas, retina, or skeletal muscle tissue.

12. The method of any one of claims 6-11, wherein the subject is an adult or a juvenile.

13. The method of any one of claims 6-11, wherein the subject is a neonate.

14. The method of any one of claims 6-11, wherein the subject is a rodent.

15. The method of any one of claims 6-14, wherein the subject is a mouse or a rat.

16. The method of any one of claims 1-15, wherein the recombinase is a Cre or Flp recombinase and the first and second target sites are Lox or FRT sites, respectively.

17. The method of claim 16, wherein the Lox or FRT sites are mutant sites for unidirectional recombination.

18. The method of any one of claims 1-15, wherein the recombinase is a phiC31 recombinase and the first and second target sites are attB and attP sites.

19. The method of any one of claims 1-18, wherein the CRISPR/Cas effector protein is Cas9.

20. The method of any one of claims 1-19, wherein the CRISPR/Cas effector protein is fused to a polypeptide functional domain that inhibits transcription (i.e., CRISPRi) or a polypeptide functional domain that activates transcription (i.e., CRISPRa).

21. The method of any one of claims 1-20, wherein the method further comprises introducing the recombinase and/or the CRISPR/Cas effector protein to the target cell, leading to said expression of the recombinase and/or the CRISPR/Cas effector protein.

22. The method of claim 21, wherein introducing the recombinase and/or the CRISPR/Cas effector protein comprises contacting the target cell with an AAV that delivers a nucleic acid encoding the recombinase and/or the CRISPR/Cas effector protein.

23. The method of any one of claims 1-22, wherein, prior to introducing said nucleic acid, the target cell comprises a nucleic acid encoding the recombinase and/or the CRISPR/Cas effector protein.

24. The method of any one of claims 1-23, wherein expression of the recombinase is controlled by a tissue specific promoter.

25. The method of any one of claims 1-23, wherein expression of the recombinase is controlled by a neuron-specific promoter.

26. The method of claim 25, wherein the neuron-specific promoter is an hSyn1 promoter.

27. The method of any one of claims 1-26, wherein the extracted DNA is episomal DNA.

28. The method of claim 2, wherein the serotype of the AAV's capsid is AAV-PHP.eB.

29. The method of any one of claims 1-28, wherein the probe sequence has a length of from 100-400 base pairs.

30. The method of any one of claims 6-29, wherein the introduction of said library comprises intravenous administration.

31. The method of any one of claims 6-29, wherein the introduction of said library comprises direct injection to an organ of the subject.

32. The method of any one of claims 6-29, wherein the introduction of said library comprises intracerebroventricular (ICV) injection.

33. The method of any one of claims 1-32, wherein the target cell is a non-dividing cell.

34. The method of any one of claims 1-32, wherein the target cell is a neuron.

35. The method of any one of claims 1-32, wherein the target cell is a brain cell.

36. A kit comprising:
(1) A nucleic acid comprising a nucleotide sequence encoding a CRISPR/Cas guide RNA and an inversion cassette that comprises a probe sequence positioned between a first and a second target site for a site-specific recombinase; and
(2) a first and a second primer, wherein the first and second primers hybridize to the same DNA strand of said nucleic acid, wherein one of the primers:
(i) hybridizes to the probe sequence such that upon inversion of the probe sequence by the recombinase, the first and second primers form a primer pair, or
(ii) hybridizes to a neo-sequence generated by excision of the probe sequence by the recombinase such that upon excision, the first and second primers form a primer pair; and
wherein the other primer hybridizes to a sequence such that the guide RNA's guide sequence is positioned between the primer pair.

37. The kit of claim 36, wherein the nucleic acid is an AAV nucleic acid vector.

38. The kit of claim 35 or claim 36, wherein the nucleotide sequence encoding the CRISPR/Cas guide RNA is operably linked to a pol Ill promoter.

39. The kit of any one of claims 36-38, wherein the probe sequence is a spacer sequence.

40. The kit of any one of claims 36-38, wherein the probe sequence comprises the sequence encoding the guide RNA.

41. The kit of any one of claims 36-40, wherein the nucleic acid further encodes a cell marker operably linked to a promoter.

42. The kit of any one of claims 36-41, wherein the recombinase is a Cre or Flp recombinase and the first and second target sites are Lox or FRT sites, respectively.

43. The kit of any one of claims 36-41, wherein the recombinase is a phiC31 recombinase and the first and second target sites are attB and attP sites.

44. The kit of any one of claims 36-43, wherein the CRISPR/Cas guide RNA is CRISPR/Cas9 guide RNA.

45. The kit of any one of claims 36-43, wherein the probe sequence has a length of from 100-400 base pairs.

46. A nucleic acid comprising:
(a) a nucleotide sequence encoding a CRISPR/Cas guide RNA,
(b) a nucleotide sequence encoding a cell marker, and
(c) an inversion cassette that comprises a probe sequence positioned between a first and a second target site for a site-specific recombinase.

47. The nucleic acid of claim 46, wherein the nucleic acid is an AAV nucleic acid vector.

48. The nucleic acid of claim 46 or claim 47, wherein the nucleotide sequence encoding the CRISPR/Cas guide RNA is operably linked to a pol III promoter.

49. The nucleic acid of any one of claims 46-48, wherein the probe sequence is a spacer sequence.

50. The nucleic acid of any one of claims 46-48, wherein the probe sequence comprises the sequence encoding the guide RNA.

51. The nucleic acid of any one of claims 46-50, wherein the recombinase is a Cre or Flp recombinase and the first and second target sites are Lox or FRT sites, respectively.

52. The nucleic acid of any one of claims 46-50, wherein the recombinase is a phiC31 recombinase and the first and second target sites are attB and attP sites

53. The nucleic acid of any one of claims 46-52, wherein the CRISPR/Cas guide RNA is CRISPR/Cas9 guide RNA.

54. The nucleic acid of any one of claims 46-53, wherein the probe sequence has a length of from 100-400 base pairs.

55. The nucleic acid of any one of claims 46-54, wherein the inversion cassette is positioned between the portion of the nucleic acid that encodes the CRISPR/Cas guide RNA and the portion of the nucleic acid that encodes the cell marker.

56. A library of the nucleic acids of any one of claims 46-55, wherein the encoded CRISPR/Cas guide RNAs of the members of said library have different guide sequences from one another.

57. A kit comprising:
(1) a library of nucleic acids, wherein each nucleic acid comprises a nucleotide sequence encoding a CRISPR/Cas guide RNA and an inversion cassette that comprises a probe sequence positioned between a first and a second target site for a site-specific recombinase, and wherein the encoded CRISPR/Cas guide RNAs of the members of said library have different guide sequences from one another; and
(2) a first and a second primer, wherein the first and second primers hybridize to the same DNA strand of said nucleic acid, wherein one of the primers:
(i) hybridizes to the probe sequence such that upon inversion of the probe sequence by the recombinase, the first and second primers form a primer pair, or
(ii) hybridizes to a neo-sequence generated by excision of the probe sequence by the recombinase such that upon excision, the first and second primers form a primer pair; and
wherein the other primer hybridizes to a sequence such that the guide RNA's guide sequence is positioned between the primer pair.


SEQUENCE LISTING (27 sequences)

SEQ ID NO: 1 - DNA, 175 bp, synthetic construct
  source 1..175: mol_type = other DNA
acatgtaaacgatcctaggcgtccatatgatgcatggcagcaattggccatttgtaggcagacgcatcgatcagtacagtcggatagtcgaacgatcattacagtcagtcgaatgcaaagtagcgaggcatgacctgtgtacttaaagcatgactggtactgacacgtcgcatgc

SEQ ID NO: 2 - DNA, 45 bp, synthetic construct
  source 1..45: mol_type = other DNA
caagcagaagacggcatacgagatgcacaaaaggaaactcaccct

SEQ ID NO: 3 - DNA, 20 bp, synthetic construct
  source 1..20: mol_type = other DNA
tgactggtactgacacgtcg

SEQ ID NO: 4 - DNA, 88 bp, synthetic construct
  source 1..88: mol_type = other DNA
aatgatacggcgaccaccgagatctacacgatcggaagagcacacgtctgaactccagtcacatcacgtgactggtactgacacgtcg

SEQ ID NO: 5 - DNA, 88 bp, synthetic construct
  source 1..88: mol_type = other DNA
aatgatacggcgaccaccgagatctacacgatcggaagagcacacgtctgaactccagtcaccgatgttgactggtactgacacgtcg

SEQ ID NO: 6 - DNA, 88 bp, synthetic construct
  source 1..88: mol_type = other DNA
aatgatacggcgaccaccgagatctacacgatcggaagagcacacgtctgaactccagtcacttaggctgactggtactgacacgtcg

SEQ ID NO: 7 - DNA, 88 bp, synthetic construct
  source 1..88: mol_type = other DNA
aatgatacggcgaccaccgagatctacacgatcggaagagcacacgtctgaactccagtcactgaccatgactggtactgacacgtcg

SEQ ID NO: 8 - DNA, 88 bp, synthetic construct
  source 1..88: mol_type = other DNA
aatgatacggcgaccaccgagatctacacgatcggaagagcacacgtctgaactccagtcacacagtgtgactggtactgacacgtcg

SEQ ID NO: 9 - DNA, 88 bp, synthetic construct
  source 1..88: mol_type = other DNA
aatgatacggcgaccaccgagatctacacgatcggaagagcacacgtctgaactccagtcacgccaattgactggtactgacacgtcg

SEQ ID NO: 10 - DNA, 88 bp, synthetic construct
  source 1..88: mol_type = other DNA
aatgatacggcgaccaccgagatctacacgatcggaagagcacacgtctgaactccagtcaccagatctgactggtactgacacgtcg

SEQ ID NO: 11 - DNA, 88 bp, synthetic construct
  source 1..88: mol_type = other DNA
aatgatacggcgaccaccgagatctacacgatcggaagagcacacgtctgaactccagtcacgtgaaatgactggtactgacacgtcg

SEQ ID NO: 12 - DNA, 88 bp, synthetic construct
  source 1..88: mol_type = other DNA
aatgatacggcgaccaccgagatctacacgatcggaagagcacacgtctgaactccagtcacgtggcctgactggtactgacacgtcg

SEQ ID NO: 13 - DNA, 88 bp, synthetic construct
  source 1..88: mol_type = other DNA
aatgatacggcgaccaccgagatctacacgatcggaagagcacacgtctgaactccagtcacgtttcgtgactggtactgacacgtcg

SEQ ID NO: 14 - DNA, 88 bp, synthetic construct
  source 1..88: mol_type = other DNA
aatgatacggcgaccaccgagatctacacgatcggaagagcacacgtctgaactccagtcacgagtggtgactggtactgacacgtcg

SEQ ID NO: 15 - DNA, 88 bp, synthetic construct
  source 1..88: mol_type = other DNA
aatgatacggcgaccaccgagatctacacgatcggaagagcacacgtctgaactccagtcacacttgatgactggtactgacacgtcg

SEQ ID NO: 16 - DNA, 88 bp, synthetic construct
  source 1..88: mol_type = other DNA
aatgatacggcgaccaccgagatctacacgatcggaagagcacacgtctgaactccagtcactagctttgactggtactgacacgtcg

SEQ ID NO: 17 - DNA, 88 bp, synthetic construct
  source 1..88: mol_type = other DNA
aatgatacggcgaccaccgagatctacacgatcggaagagcacacgtctgaactccagtcacggctactgactggtactgacacgtcg

SEQ ID NO: 18 - DNA, 88 bp, synthetic construct
  source 1..88: mol_type = other DNA
aatgatacggcgaccaccgagatctacacgatcggaagagcacacgtctgaactccagtcaccttgtatgactggtactgacacgtcg

SEQ ID NO: 19 - DNA, 88 bp, synthetic construct
  source 1..88: mol_type = other DNA
aatgatacggcgaccaccgagatctacacgatcggaagagcacacgtctgaactccagtcacagtcaatgactggtactgacacgtcg

SEQ ID NO: 20 - DNA, 88 bp, synthetic construct
  source 1..88: mol_type = other DNA
aatgatacggcgaccaccgagatctacacgatcggaagagcacacgtctgaactccagtcacagttcctgactggtactgacacgtcg

SEQ ID NO: 21 - DNA, 88 bp, synthetic construct
  source 1..88: mol_type = other DNA
aatgatacggcgaccaccgagatctacacgatcggaagagcacacgtctgaactccagtcacactgattgactggtactgacacgtcg

SEQ ID NO: 22 - DNA, 88 bp, synthetic construct
  source 1..88: mol_type = other DNA
aatgatacggcgaccaccgagatctacacgatcggaagagcacacgtctgaactccagtcacattccttgactggtactgacacgtcg

SEQ ID NO: 23 - DNA, 303 bp, Mus musculus
  source 1..303: mol_type = genomic DNA
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggaga

SEQ ID NO: 24 - DNA, 11 bp, unidentified
  source 1..11: mol_type = unassigned DNA
ccaccttgttg

SEQ ID NO: 25 - DNA, 20 bp, synthetic construct
  source 1..20: mol_type = other DNA
ggctgcggctcctcagtcgg

SEQ ID NO: 26 - DNA, 20 bp, synthetic construct
  source 1..20: mol_type = other DNA
gaacactgacctggacactt

SEQ ID NO: 27 - DNA, 21 bp, synthetic construct
  source 1..21: mol_type = other DNA
ggatgcatagatgaacggatg